SEQUENCE LISTING (10 sequences)

SEQ ID NO: 1 - DNA, 20 bp, Artificial Sequence
  synthetic oligonucleotide
caaatgggctccctctcatc

SEQ ID NO: 2 - DNA, 20 bp, Artificial Sequence
  synthetic oligonucleotide
gctcctctgcttggtggttt

SEQ ID NO: 3 - DNA, 20 bp, Artificial Sequence
  synthetic oligonucleotide
aaggtccggtccatcttgtg

SEQ ID NO: 4 - DNA, 20 bp, Artificial Sequence
  synthetic oligonucleotide
atccttcactgcaccctcgt

SEQ ID NO: 5 - DNA, 20 bp, Artificial Sequence
  synthetic oligonucleotide
ctgggacgatatggagaaga

SEQ ID NO: 6 - DNA, 20 bp, Artificial Sequence
  synthetic oligonucleotide
accagaggcatacagggaca

SEQ ID NO: 7 - DNA, 21 bp, Artificial Sequence
  synthetic oligonucleotide
tggtctttgggaaggtgaaag

SEQ ID NO: 8 - DNA, 20 bp, Artificial Sequence
  synthetic oligonucleotide
tgtccacagtcggaaatggt

SEQ ID NO: 9 - DNA, 20 bp, Artificial Sequence
  synthetic oligonucleotide
cagtcaacgggggacataaa

SEQ ID NO: 10 - DNA, 21 bp, Artificial Sequence
  synthetic oligonucleotide
agaggtccttttcaccagcaa

(19) United States
(12) Patent Application Publication
(10) Pub. No.: US 2010/0261666 A1
(43) Pub. Date: Oct. 14, 2010

(54) COMPOSITIONS AND METHODS FOR THE TREATMENT OF MYOCARDIAL DYSFUNCTION ASSOCIATED WITH SIRS OR SEPSIS

(75) Inventors: James D. Ross, San Antonio, TX (US); William R. Law, Wayne, PA (US)

Correspondence Address: SCHWABE, WILLIAMSON & WYATT, P.C., 1420 FIFTH, SUITE 3400, SEATTLE, WA, 98101-4010, US

(73) Assignee: THE BOARD OF TRUSTEES OF THE UNIVERSITY OF ILLINOIS, URBANA, IL (US)

(21) Appl. No.: 12/760,381

(22) Filed: Apr. 14, 2010

Related U.S. Application Data
(60) Provisional application No. 61/169,233, filed on Apr. 14, 2009.

(51) Int. Cl. A61K 31/7064; A61K 31/519; A61P 9/00

(52) U.S. Cl. 514/43; 514/262.1

(57) ABSTRACT

Provided are compositions and methods for the treatment of myocardial dysfunction associated with SIRS or sepsis, which methods comprise the administration to a patient in need thereof of a composition comprising one or more adenosine deaminase (ADA) inhibitor and/or one or more xanthine oxidase (XO) inhibitor. Exemplified herein are methods for the treatment of myocardial dysfunction, which methods comprise the administration of a composition comprising the ADA inhibitor pentostatin and/or a composition comprising the XO inhibitor allopurinol. Advantageously, the methods disclosed herein that employ the administration of one or more ADA inhibitor(s) do not significantly affect cardiac TNF-α mRNA expression and/or protein levels.

### CROSS REFERENCE TO RELATED APPLICATIONS

This application claims the benefit of U.S. Provisional Patent Application No. 61/169,233, filed Apr. 14, 2009 the entire disclosure of which is hereby incorporated by reference in its entirety.

### BACKGROUND OF THE DISCLOSURE

1. Technical Field

The present disclosure is directed, generally, to the treatment of myocardial dysfunction. More specifically, disclosed herein are compositions and methods for the treatment of myocardial dysfunction, which methods comprise the administration of one or more adenosine deaminase inhibitor(s), such as pentostatin (a/k/a deoxycoformycin, dCF), and/or one or more xanthine oxidase inhibitor(s), such as allopurinol.

2. Description of the Related Art

Depressed myocardial function is associated with many cardiovascular disorders such as heart failure, ischemia-reperfusion injury, and sepsis [1-19]. Sepsis is defined as the presence of a confirmed infection and the resultant systemic inflammatory response syndrome (SIRS) [28]. With a reported mortality rate ranging from 28-50%, sepsis is the 10th leading cause of death in the United States [29, 30]. Alterations in cardiac function resulting in poor tissue oxidation and organ failure are likely the primary cause of death [29, 31].

Septic patients may display a complex variety of cardiovascular derangements. The regulation of cardiovascular function and dysfunction in sepsis is complex and poorly understood. Studies have indicated, however, that intrinsic myocardial function is altered in sepsis, and that sepsis-induced cardiac dysfunction may be reversible [36-40].

Tumor necrosis factor α (TNF-α) may play a significant role in the modulation of myocardial dysfunction associated with sepsis. Elevations in TNF-α are associated with the aforementioned cardiovascular disorders [20], and TNF-α exerts a negative inotropic effect on the myocardium [3, 7, 21]. TNF-α plays beneficial roles in innate immunity, hematopoiesis, and organogenesis [43-45]. Conversely, in acute infection and inflammation, increased levels of TNF-α are associated with shock, fever, respiratory arrest, capillary leak syndrome, hemorrhagic necrosis, and lactic acidosis [42]. In the inflammatory response, TNF-α may function as a chemoattractant, activate leukocytes, enhance nonspecific host resistance and induce the formation of reactive oxygen intermediates (ROI) [42].

TNF-α has been characterized as a myocardial depressant (i.e., depresses left ventricular ejection fraction and alters the Frank-Starling and diastolic pressure-volume relationship) [21, 46-50]. Physiological derangements including hypotension have been observed in rats and dogs after administration of TNF-α [47, 48]. In dogs, mean arterial blood pressure was significantly reduced in response to recombinant TNF infusion; left ventricular ejection fraction was also significantly decreased, even after volume infusion [50]. TNF-α elevation has been observed in myocardial ischemia-reperfusion models [51-53]. TNF-α has also been correlated with the progression of chronic heart failure [9, 11, 19, 56] and cardiac allograft rejection [15, 20, 57-59]. TNF-α inhibition has been shown to protect cardiac function in models of ischemia-reperfusion [54, 55].

Resident cardiac macrophages and cardiac myocytes produce TNF-α. Cardiac myocytes have been shown to contribute to TNF-α mRNA expression and protein production in response to endotoxin [22]. Monocytic cells of feline hearts stimulated with lipopolysachamide (LPS) displayed significant elevations in TNF-α mRNA expression that progressed in a time-dependent manner, peaking at 90 minutes. TNF-α expression subsequently declined but remained elevated in comparison to controls for as long as 210 minutes after LPS stimulation. Cardiac myocytes were observed to produce as much TNF-α protein as the non-myocyte cells. [22].

Bacterial membrane LPS triggers a complex signaling cascade that results in the production of TNF-α in the heart [4, 22, 60]. LPS binds lipopolysachamide binding protein, which complexes with membrane-associated CD14. This association triggers the rapid intracellular tyrosine phosphorylation of Ras, which initiates a protein kinase cascade, resulting in TNF-α production [5]. Myocyte and resident macrophage-derived TNF-α contribute to myocardial dysfunction in two phases, an initial phase and a nitric oxide (NO) dependent late phase [61]. Immediate negative inotropic effects of TNF-α are mediated by sphingosine in feline cardiac myocytes [62]. This immediate mechanism of TNF-α induced cardiac depression is independent of NO. The second, or late phase, of TNF-α induced myocardial dysfunction is described as NO-dependent [5]. Inducible nitric oxide synthase levels become elevated, resulting in increased NO production which subsequently acts to desensitize the myofilaments to calcium [63]. This desensitization results in a sustained contractile dysfunction [64].

While there is a great deal of evidence that TNF-α contributes to myocardial contractile dysfunction in various cardiovascular disorders, the mechanisms by which TNF-α mRNA expression and protein production are regulated in the heart remain unclear. Both adenosine and reactive oxygen species (ROS) have, however, been implicated in the regulation of myocardial TNF-α. Adenosine reduces cardiac TNF-α production while ROS elicit the opposite effect. These two effectors are linked by the enzyme adenosine deaminase (ADA), which converts adenosine to inosine that, in turn, is further metabolized to form hypoxanthine. Xanthine oxidase (XO) converts hypoxanthine to xanthine and xanthine to uric acid, and both XO-catalyzed steps result in the production of reactive oxygen species (ROS). ROS (specifically in the form of hydrogen peroxide) may promote the production of TNF-α [26, 60, 65-67].

ROS are implicated in the promotion of TNF-α production [26, 60, 67, 76, 77]. XO activity modulates the production of ROS. XO production of ROS is dependent upon the availability of substrate provided by the metabolism of adenosine to inosine (i.e., by ADA) and further metabolism of inosine to hypoxanthine. Thus, elevation of ADA activity may lead to increased degradation of adenosine downstream, increased production of substrates required for the production of ROS, and a resulting increase in TNF-α mRNA expression and protein production. The catabolic actions of XO result in the production of ROS in the form of hydrogen peroxide that can promote cardiac TNF-α production.

There is some evidence that ADA may contribute to cardiac dysfunction, and that ADA inhibition may have a protective effect in ischemia [79-81]. A protective effect for ADA inhibition in the systemic inflammatory response has also been described [27, 82]. Studies of systemic inflammatory response also demonstrate a protective role for ADA inhibition and a correlation between ADA activity and TNF-α production.

What is critically needed in the art are compositions and methods for treating myocardial dysfunction in a patient.

### SUMMARY OF THE DISCLOSURE

The present disclosure achieves these and other related needs by providing compositions and methods for the treatment of myocardial dysfunction associated with sepsis and/or SIRS. Unexpectedly, it was found as part of the present disclosure that administration of the adenosine deaminase (ADA) inhibitor pentostatin and/or the Xanthine Oxidase (XO) inhibitor allopurinol improved indices of cardiac dysfunction and pentostatin does so without significantly affecting cardiac TNF-α mRNA expression. Thus, the use of an ADA inhibitor and an XO inhibitor, separately or in combination, may be advantageously, and quite surprisingly, employed in compositions and methods for treating SIRS- and sepsis-associated myocardial dysfunction as is described in greater detail herein.

Thus, within certain embodiments, the present disclosure provides methods for the treatment of myocardial dysfunction associated with SIRS and sepsis, which methods comprise the step of administering a composition comprising one or more adenosine deaminase (ADA) inhibitor(s), such as an ADA-1 inhibitor and/or an ADA-2 inhibitor. In particular, one or more inhibitors of ADA, such as 2′-deoxycoformycin (dCF) (a/k/a pentostatin), coformycin, and/or an analog or derivative thereof, may be used to treat or prevent such a condition.

Within other embodiments, the present disclosure provides methods for the treatment of myocardial dysfunction associated with SIRS and sepsis, which methods comprise the step of administering a composition comprising one or more xanthine oxidase (XO) inhibitor(s). In particular, one or more inhibitor(s) of XO, such as allopurinol, and/or an analog or derivative thereof, may be used to treat or prevent such a condition.

Within still further embodiments, the present disclosure provides methods for the treatment of myocardial dysfunction associated with SIRS and sepsis, which methods comprise the step of administering a composition comprising a combination of one or more ADA inhibitor(s), such as dCF (pentostatin), and one or more XO inhibitor(s), such as allopurinol, and/or one or more analog(s) or derivative(s) of dCF or of allopurinol.

The present disclosure also provides compositions that may be employed in the treatment of myocardial dysfunction associated with SIRS and sepsis, which compositions comprise one or more ADA inhibitor(s) and one or more XO inhibitor(s).

### BRIEF DESCRIPTION OF THE FIGURES

In the following detailed description, reference is made to the accompanying figures.

FIG. 1 is a schema of adenosine metabolism and TNF-α regulation.

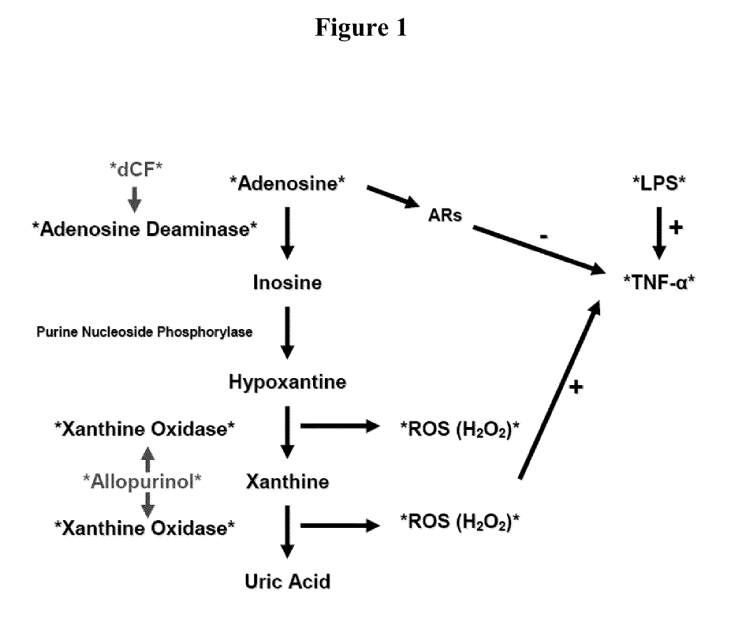

FIG. 2 is a graph depicting six day septic rat survival. Sham (rats that received surgery but not treated), Sepsis-NoRx (septic rats that received no treatment), Sepsis-dCF(Post) (rats that received 1.0 mg/kg dCF at 1 hour after the induction of sepsis) and Sepsis-dCF(Pre) (rats that received 1.0 mg/kg dCF at 1 day prior to induction of sepsis). Six day survival is presented as the percent of rats in each group surviving at 1, 3, and 6 days.

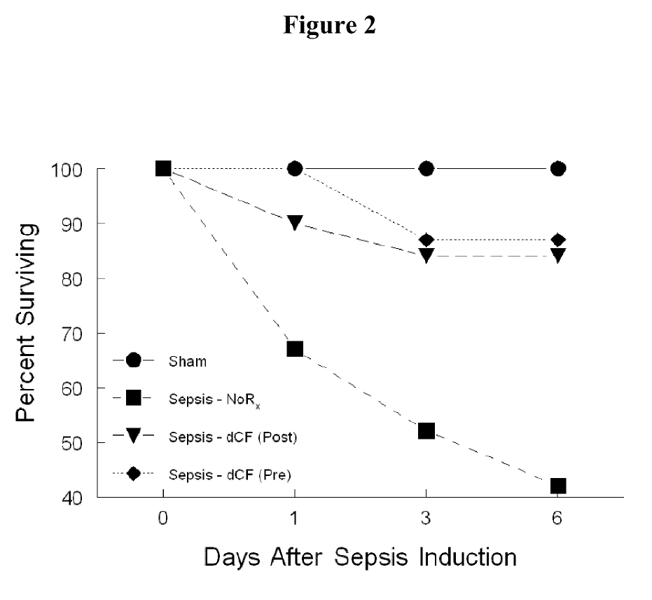

FIG. 3 is a graph depicting septic rat cardiac adenosine deaminase activity. Septic-NoRx (cardiac ADA activity in rats that were not treated before induction of sepsis) and Septic-dCF (cardiad ADA activity in rats that were pre-treated with dCF (1.0 mg/kg) before induction of sepsis). Values are presented as mean±SD cardiac ADA activity in nmol ammonia produced per hour per microgram protein.

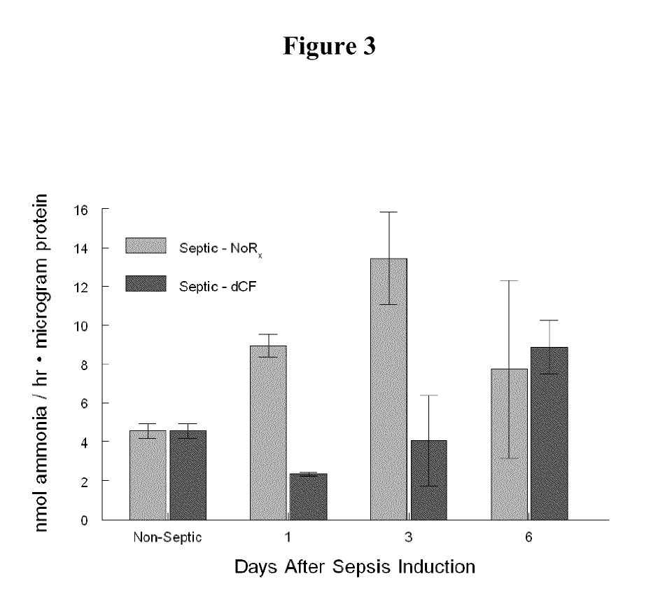

FIG. 4 is a graph depicting a time course of CON and LPS left ventricular pressures. FIG. 4 (Panel A) is CON (control, n=8, vehicle pre-treatment, vehicle infusion) left ventricular pressure time course; FIG. 4 (Panel B) is LPS (lipopolysachamide, n=8, vehicle pre-treatment, LPS infusion) left ventricular pressure time course.

FIG. 5 is a graph depicting change in 30 and 150 minute left ventricular systolic pressure. CON (control, n=8, vehicle pre-treatment, vehicle infusion), LPS (lipopolysachamide, n=8, vehicle pre-treatment, LPS infusion), dCF-CON (2′-deoxycoformycin-control), n=6, dCF pre-treatment, vehicle infusion) and dCF-LPS (2′-deoxycoformycin-lipopolysachamide, n=6, dCF pre-treatment, LPS infusion) change in LVsysP at 30 and 150 minutes. Values are expressed as mean±SEM left ventricular pressure in mmHg. (*) designates a statistically significant difference from CON. (**) designates a statistically significant difference from dCF-CON. (‡) designates a statistically significant difference from baseline (0 minutes) value.

FIG. 6 is a graph depicting change in 30 and 150 minute left ventricular developed pressure. CON (control, n=8, vehicle pre-treatment, vehicle infusion), LPS (lipopolysachamide, n=8, vehicle pre-treatment, LPS infusion), dCF-CON (2′-deoxycoformycin-control), n=6, dCF pre-treatment, vehicle infusion), and dCF-LPS (2′-deoxycoformycin-lipopolysachamide, n=6, dCF pre-treatment, LPS infusion) change in LVdevP at 30 and 150 minutes. Values are expressed as mean±SEM left ventricular pressure in mmHg. (*) designates a statistically significant difference from CON. (**) designates a statistically significant difference from dCF-CON. (†) designates a statistically significant difference from LPS. (‡) designates a statistically significant difference from baseline (0 minutes) value.

FIG. 7 is a graph depicting change in 30 and 150 minute +dP/dt. CON (control, n=8, vehicle pre-treatment, vehicle infusion), LPS (lipopolysachamide, n=8, vehicle pre-treatment, LPS infusion), dCF-CON (2′-deoxycoformycin-control), n=6, dCF pre-treatment, vehicle infusion), and dCF-LPS (2′-deoxycoformycin-lipopolysachamide, n=6, dCF pre-treatment, LPS infusion) change in +dP/dt at 30 and 150 minutes. Values are expressed as mean±SEM left ventricular pressure in mmHg. (*) designates a statistically significant difference from CON. (**) designates a statistically significant difference from dCF-CON. (†) designates a statistically significant difference from LPS. (‡) designates a statistically significant difference from baseline (0 minutes) value.

FIG. 8 is a graph depicting change in 30 and 150 minute left ventricular diastolic pressure. CON (control, n=8, vehicle pre-treatment, vehicle infusion), LPS (lipopolysachamide, n=8, vehicle pre-treatment, LPS infusion), dCF-CON (2′-deoxycoformycin-control), n=6, dCF pre-treatment, vehicle infusion), and dCF-LPS (2′-deoxycoformycin-lipopolysachamide, n=6, dCF pre-treatment, LPS infusion) change in LVdiaP at 30 and 150 minutes. Values are expressed as mean±SEM left ventricular pressure in mmHg. (*) designates a statistically significant difference from CON. (**) designates a statistically significant difference from dCF-CON. (†) designates a statistically significant difference from LPS. (‡) designates a statistically significant difference from baseline (0 minutes).

FIG. 9 is a graph depicting change in 30 and 150 minute −dP/dt. CON (control, n=8, vehicle pre-treatment, vehicle infusion), LPS (lipopolysachamide, n=8, vehicle pre-treatment, LPS infusion), dCF-CON (2′-deoxycoformycin-control), n=6, dCF pre-treatment, vehicle infusion), and dCF-LPS (2′-deoxycoformycin-lipopolysachamide, n=6, dCF pre-treatment, LPS infusion) change in −dP/dt at 30 and 150 minutes. Values are expressed as mean±SEM left ventricular pressure in mmHg. (*) designates a statistically significant difference from CON. (**) designates a statistically significant difference from dCF-CON. (‡) designates a statistically significant difference from baseline (0 minutes).

FIG. 10 is a graph depicting left ventricular adenosine deaminase activity. CON (control, n=8, vehicle pre-treatment, vehicle infusion), LPS (lipopolysachamide, n=8, vehicle pre-treatment, LPS infusion), dCF-CON (2′-deoxycoformycin-control), n=6, dCF pre-treatment, vehicle infusion), and dCF-LPS (2′-deoxycoformycin-lipopolysachamide, n=6, dCF pre-treatment, LPS infusion) left ventricular adenosine deaminase activity. (*) indicates a statistically significant difference from CON (p<0.05). Values are expressed as mean±SEM. (†) indicates a statistically significant difference from LPS (p<0.05).

FIG. 11 is a graph depicting left ventricular ADA1 mRNA expression. CON (control, n=8, vehicle pre-treatment, vehicle infusion), LPS (lipopolysachamide, n=8, vehicle pre-treatment, LPS infusion), dCF-CON (2′-deoxycoformycin-control), n=6, dCF pre-treatment, vehicle infusion), and dCF-LPS (2′-deoxycoformycin-lipopolysachamide, n=6, dCF pre-treatment, LPS infusion) left ventricular ADA1 mRNA expression. Values are expressed as mean±SEM.

FIG. 12 is a graph depicting left ventricular TNF-α mRNA expression. CON (control, n=8, vehicle pre-treatment, vehicle infusion), LPS (lipopolysachamide, n=8, vehicle pre-treatment, LPS infusion), dCF-CON (2′-deoxycoformycin-control), n=6, dCF pre-treatment, vehicle infusion), and dCF-LPS (2′-deoxycoformycin-lipopolysachamide, n=6, dCF pre-treatment, LPS infusion) left ventricular TNF-α mRNA expression. Values are expressed as mean±SEM. (*) indicates a statistically significant difference from CON (p<0.05). (**) indicates a statistically significant difference from dCF-CON.

FIG. 13 is a graph depicting change in 30 and 150 minute left ventricular systolic pressure. CON (control, n=8, vehicle pre-treatment, vehicle infusion), LPS (lipopolysachamide, n=8, vehicle pre-treatment, LPS infusion), ALO-CON (Allopurinol-control), n=6, ALO pre-treatment, vehicle infusion), and ALO-LPS (Allopurinol-lipopolysacharride, n=6, ALO pre-treatment, LPS infusion) change in LVsysP at 30 and 150 minutes. Values are expressed as mean±SEM left ventricular pressure in mmHg. (*) designates a statistically significant difference from CON. (**) designates a statistically significant difference from ALO-CON. (‡) designates a statistically significant difference from baseline (0 minutes).

FIG. 14 is a graph depicting change in 30 and 150 minute left ventricular developed pressure. CON (control, n=8, vehicle pre-treatment, vehicle infusion), LPS (lipopolysachamide, n=8, vehicle pre-treatment, LPS infusion), ALO-CON (Allopurinol-control), n=6, ALO pre-treatment, vehicle infusion), and ALO-LPS (Allopurinol-lipopolysachamide, n=6, ALO pre-treatment, LPS infusion) change in LVdevP at 30 and 150 minutes. Values are expressed as mean±SEM left ventricular pressure in mmHg. (*) designates a statistically significant difference from CON. (**) designates a statistically significant difference from ALO-CON. (‡) designates a statistically significant difference from baseline (0 minutes).

FIG. 15 is a graph depicting change in 30 and 150 minute +dP/dt. CON (control, n=8, vehicle pre-treatment, vehicle infusion), LPS (lipopolysachamide, n=8, vehicle pre-treatment, LPS infusion), ALO-CON (Allopurinol-control), n=6, ALO pre-treatment, vehicle infusion), and ALO-LPS (Allopurinol-lipopolysachamide, n=6, ALO pre-treatment, LPS infusion) change in +dP/dt at 30 and 150 minutes. Values are expressed as mean±SEM. (*) designates a statistically significant difference from CON. (**) designates a statistically significant difference from ALO-CON. (‡) designates a statistically significant difference from baseline (0 minutes).

FIG. 16 is a graph depicting change in 30 and 150 minute left ventricular diastolic pressure. CON (control, n=8, vehicle pre-treatment, vehicle infusion), LPS (lipopolysachamide, n=8, vehicle pre-treatment, LPS infusion), ALO-CON (Allopurinol-control), n=6, ALO pre-treatment, vehicle infusion), and ALO-LPS (Allopurinol-lipopolysachamide, n=6, ALO pre-treatment, LPS infusion) change in LVdiaP at 30 and 150 minutes. Values are expressed as mean±SEM left ventricular pressure in mmHg. (*) designates a statistically significant difference from CON. (**) designates a statistically significant difference from ALO-CON. (†) designates a statistically significant difference from LPS. (‡) designates a statistically significant difference from baseline (0 minutes).

FIG. 17 is a graph depicting change in 30 and 150 minute −dP/dt. CON (control, n=8, vehicle pre-treatment, vehicle infusion), LPS (lipopolysachamide, n=8, vehicle pre-treatment, LPS infusion), ALO-CON (Allopurinol-control), n=6, ALO pre-treatment, vehicle infusion), and ALO-LPS (Allopurinol-lipopolysachamide, n=6, ALO pre-treatment, LPS infusion) change in −dP/dt at 30 and 150 minutes. Values are expressed as mean±SEM left ventricular pressure in mmHg. (*) designates a statistically significant difference from CON. (**) designates a statistically significant difference from ALO-CON. (‡) designates a statistically significant difference from baseline (0 minutes).

FIG. 18 is a graph depicting left ventricular adenosine deaminase activity. CON (control, n=8, vehicle pre-treatment, vehicle infusion), LPS (lipopolysachamide, n=8, vehicle pre-treatment, LPS infusion), ALO-CON (Allopurinol-control), n=6, ALO pre-treatment, vehicle infusion), and ALO-LPS (Allopurinol-lipopolysachamide, n=6, ALO pre-treatment, LPS infusion) left ventricular ADA activity. Values are expressed as mean±SEM.

FIG. 19 is a graph depicting left ventricular ADA1 mRNA expression. CON (control, n=8, vehicle pre-treatment, vehicle infusion), LPS (lipopolysachamide, n=8, vehicle pre-treatment, LPS infusion), ALO-CON (Allopurinol-control), n=6, ALO pre-treatment, vehicle infusion), and ALO-LPS (Allopurinol-lipopolysachamide, n=6, ALO pre-treatment, LPS infusion) left ventricular ADA1 mRNA expression. Values are expressed as mean±SEM.

FIG. 20 is a graph depicting left ventricular TNF-α mRNA expression. CON (control, n=8, vehicle pre-treatment, vehicle infusion), LPS (lipopolysachamide, n=8, vehicle pre-treatment, LPS infusion), ALO-CON (Allopurinol-control), n=6, ALO pre-treatment, vehicle, infusion), and ALO-LPS (Allopurinol-lipopolysachamide, n=6, ALO pre-treatment, LPS infusion) left ventricular TNF-α mRNA expression. Values are expressed as mean±SEM. (*) indicates a statistically significant difference from CON. (**) indicates a statistically significant difference from ALO-CON. (†) indicates a statistically significant difference from LPS.

### DETAILED DESCRIPTION OF THE DISCLOSURE

The present disclosure is based upon the unexpected discovery that the adenosine deaminase (ADA) inhibitor pentostatin (a/k/a deoxycoformycin, dCF) and/or the xanthine oxidase (XO) inhibitor allopurinol may be advantageously employed in compositions and methods for the treatment of a patient with myocardial dysfunction associated with SIRS or sepsis. Quite surprisingly, it was found that administration of ADA and XO inhibitors are effective in reducing or preventing one or more indicia of myocardial dysfunction and ADA inhibitors do so without significantly affecting in vivo levels of TNF-α in the patient. While the present disclosure exemplifies methods for the treatment of myocardial dysfunction that employ pentostatin or allopurinol, it will be understood that alternative ADA and/or XO inhibitors, including analogs and derivatives of pentostatin (dCF) and allopurinol, may also be suitably employed in these methods.

As discussed above, it is believed that adenosine inhibits LPS-stimulated cardiac TNF-α production, that ROS may stimulate cardiac TNF-α production, that ADA is intimately tied to both adenosine and ROS production through adenosine metabolism, and that ADA activity is elevated during systemic inflammatory responses. Prior to the discoveries presented herein, however, the contribution of ADA in the cardiac response to an inflammatory challenge remained unknown.

In stark contrast to what has been suggested by earlier reports of ADA inhibition in other tissue types and disease models wherein elevation of ADA activity led to elevated TNF-α mRNA expression and protein production by way of elevated downstream adenosine degradation and production of ROS substrates, ADA inhibition with an ADA inhibitor such as dCF does not reduce the expression of myocardial TNF-α mRNA.

The influence of ADA and XO on LPS-challenged cardiac dysfunction was investigated in the context of left ventricular mechanical performance and TNF-α mRNA expression. The results indicated that administration of dCF to patients with SIRS/sepsis would be effective in reducing, ameliorating, and/or preventing cardiac dysfunction associated with those conditions. Contrary to earlier reports, ADA inhibition may be protective due to its effects on cardiac adenosine levels, rather than through reduction of cardiac TNF-α mRNA expression.

Thus, and without being limited to mechanistic theory, it appears that the ADA inhibitor pentostatin (dCF) inhibits ADA activity and protects cardiac function under conditions of SIRS and sepsis without significantly affecting the expression of myocardial TNF-α. These unexpected results suggest, in contrast to previous findings for other tissue types and disease models, that myocardial ADA does not affect LPS-induced cardiac dysfunction through the regulation of myocardial TNF-α mRNA expression. Instead, myocardial ADA may play a different role in sepsis- and SIRS-related myocardial dysfunction.

The present disclosure will be best understood by reference to the following definitions:

### DEFINITIONS

As used herein, the term “SIRS” refers to “systemic inflammatory response syndrome,” which is an inflammatory state affecting the whole body. As used herein, the term “sepsis” refers to a medical condition that is characterized by a whole-body inflammatory state and the presence of a known or suspected infection.

As used herein, the term “myocardial dysfunction” refers to a cardiac disease state that is associated with SIRS and sepsis and can be characterized by a hyperdynamic state, with tachycardia, normal-to-low blood pressure, normal-to-high cardiac index, low systemic vascular resistance, and diastolic dysfunction, which can severely impair myocardial performance. Classic indicia of myocardial dysfunction include any combination of depressed left ventricular velocity of contraction and relaxation, coronary flow, and decreases in left ventricular systolic (LV developed pressure, +dP/dt) and diastolic (LV diastolic pressure) pressures, evidenced clinically by decreases in cardiac output and/or decreases in cardiac ejection fraction.

As used herein, the term “adenosine deaminase (ADA)” refers to a class of enzymes involved in purine metabolism that deaminate adenosine thereby converting it to inosine. Two isoforms of adenosinde deaminase have been identified: ADA1 and ADA2. ADA1 is ubiquitously expressed, especially in lymphocytes and macrophages. ADA2 was first identified in human spleen, but has also been identified in other tissues including macrophages where it is co-expressed with ADA1.

As used herein, the term “xanthine oxidase” refers to an enzyme that catalyzes the oxidation of hypoxanthine to xanthine and xanthine to uric acid.

As used herein, the term “analog” refers to a compound that is structurally similar to a parent compound (e.g., the ADA inhibitor pentostatin or the XO inhibitor allopurinol), but differs slightly in composition (e.g., one atom or functional group is different, added, or removed). Analogs typically possess similar chemical, biological, and/or physical properties as compared to the parent compound from which it is derived.

As used herein, the term “alkyl” refers to saturated straight- or branched-chain aliphatic groups containing from 1-20 carbon atoms, typically 1-8 carbon atoms or 1-4 carbon atoms. This definition applies as well to the alkyl portion of alkoxy, alkanoyl and aralkyl groups.

As used herein, the term “alkoxy” refers to alkyl groups covalently linked to an oxygen atom. Alkoxy groups typically contain between 1 and 10 carbon atoms. For example, alkoxy groups include, but are not limited to, methoxy, ethoxy, isopropyloxy, propoxy, butoxy, and pentoxy groups.

As used herein, the term “amino” as used herein refers to the group —NRR, wherein R may independently be hydrogen, alkyl, aryl, alkoxy, or heteroaryl.

As used herein, the term “therapeutically effective amount” means an amount of an ADA or an XO inhibitor that is sufficient to result in a decrease in severity of one or more indices of myocardial dysfunction in the patient to which it is administered. One of ordinary skill in the art would be able to determine such therapeutically effective amounts based on such factors as the subject's size, the severity of symptoms, and the particular composition or route of administration selected. Inhibitory compounds of the present disclosure, individually, or in combination or in conjunction with other drugs, can be used to treat myocardial dysfunction associated with SIRS and/or sepsis as discussed herein.

Methods for the Treatment of Myocardial Dysfunction

As described above, the present disclosure provides methods for the treatment of myocardial dysfunction that is associated with SIRS and/or sepsis in a patient. These methods comprise the step of administering to a patient in need thereof a composition comprising one or more adenosine deaminase (ADA) inhibitor(s) and/or one or more xanthine oxidase (XO) inhibitor(s) at a time and dosage sufficient to achieve substantial improvement in one or more indicia of myocardial dysfunction.

These methods are based upon the unexpected finding that the administration of an ADA inhibitor is effective in improving or preventing one or more indicia of myocardial dysfunction without substantially affect the in vivo levels of cardiac TNF-α in a patient suffering from, or predicted to be afflicted with, myocardial dysfunction that is attributed to SIRS and/or sepsis.

Myocardial dysfunction associated with SIRS and sepsis is reviewed in Sharma, Shock 28(3):265-269 (2007). Indicia of myocardial dysfunction can include a hyperdynamic state, with tachycardia, normal-to-low blood pressure, normal-to-high cardiac index, low systemic vascular resistance, and diastolic dysfunction. See, also, Hollenbergi et al., Crit. Care Med 32:1928-1948 (2004). Increased heart rate and regional vascular alterations can severely impair myocardial performance. Classic indicia of myocardial dysfunction, which are exemplified herein, include depressed left ventricular velocity of contraction and relaxation, coronary flow, and decreases in left ventricular systolic (LV developed pressure, +dP/dt) and diastolic (LV diastolic pressure) pressures as evidenced clinically by decreases in cardiac output and/or left ventricular ejection fraction. See Braun-Duallaeus et al. [88], Stamm et al., [89], and Farias et al., [90].

Within certain embodiments, the presently disclosed methods for the treatment of myocardial dysfunction comprise the administration of one or more adenosine deaminase (ADA) inhibitor(s) that is a compound of Formula I:

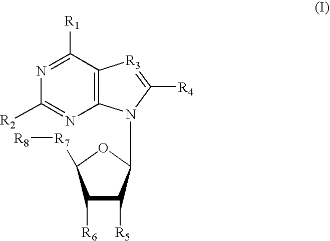

wherein

R₁ and R₂ are independently selected from the group consisting of H, OH, NH₂, OCH₃, CH₃, amino, amide, alkyl, alkoxyl, sulfhydryl, alkylthio, halogen, nitryl, phosphoryl, sulfinyl, and sulfonyl;

R₃ is selected from the group consisting of CH, N, or an acyclic substituent,

R₄ is selected from the group consisting of H, OH, halogen, alkyl; alkoxyl, amino, amide, sulfhydryl, nitryl, phosphoryl, sulfinyl, and sulfonyl;

R₅ is selected from the group consisting of H, OH, and halogen;

R₆ is selected from the group consisting of H, OH, and halogen;

R₇ is selected from the group consisting of CH₂ and phosphoryl; and

R₈ is selected from the group consisting of H, OH, amino, alkoxy, alkyl, and phosphoryl.

Exemplary compounds of Formula I that may be employed in these methods include cladribine, fludarabine, nelarabine, clofarabine, and vidarabine.

Within other embodiments, the present methods for the treatment of myocardial dysfunction comprise the administration of one or more adenosine deaminase (ADA) inhibitor(s) that is a compound of Formula II:

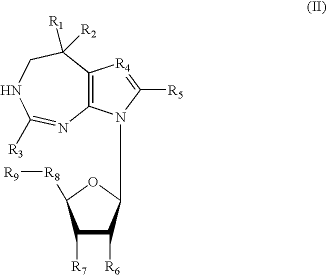

wherein

R₁, R₂, R₃, and R₅ are each independently selected from the group consisting of H, OH, NH₂, OCH₃, CH₃, amino, amide, alkyl, alkoxyl, sulfhydryl, alkylthio, halogen, nitryl, phosphoryl, sulfinyl, and sulfonyl;

R₄ is selected from the group consisting of CH, N, and an acyclic substituent such as CH—O—CH(COOH)₂;

R₆ is selected from the group consisting of halogen, H, and OH;

R₇ is selected from the group consisting of halogen, H, and OH;

R₈ is selected from the group consisting of CH₂ and phosphoryl; and

R₉ is selected from the group consisting of H, OH, amino, alkoxy, alkyl, and phosphoryl.

Exemplary compounds of Formula II that may be employed in these methods include, from left to right, 2′-deoxy-8-epi-2′-fluorocoformycin, coformycin, 2-chloropentostatin, and 2′-deoxycoformycin (dCF, pentostatin):

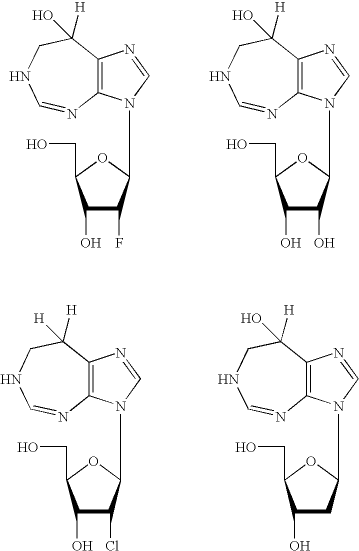

Within other embodiments, the present methods for the treatment of myocardial dysfunction comprise the administration of one or more adenosine deaminase (ADA) inhibitor(s) that is a compound of Formula III:

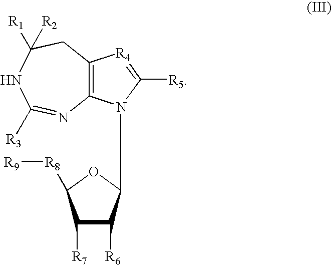

wherein

R₁, R₂, R₃, and R₅ are each independently selected from the group consisting of H, OH, amino, amide, alkyl, alkylthio, alkoxy, halogen, sulfhydryl, nitryl, phosphoryl, and sulfonyl;

R₄ is selected from the group consisting of CH, N, and an acyclic substituent such as CH—O—CH(COOH)₂;

R₆ is selected from the group consisting of halogen, H, and OH;

R₇ is selected from the group consisting of halogen, H, and OH;

R₈ is selected from the group consisting of CH₂ and phosphoryl; and

R₉ is selected from the group consisting of H, OH, amino, alkoxy, alkyl, and phosphoryl.

An exemplary compound of Formula III that may be employed in these methods is isocoformycin:

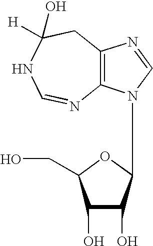

Within still further embodiments, the presently disclosed methods for the treatment of myocardial dysfunction comprise the administration of one or more xanthine oxidase (XO) inhibitor(s) that is a compound of Formula IV:

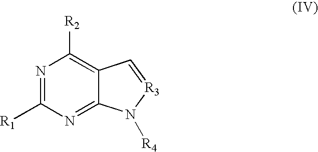

wherein

R₁ and R₂ are each independently selected from the group consisting of H, OH, O, S, halogen, mercapto, cyano, methylamine, hydro'carbon, amino, amide, alkyl, alkylthio, alkoxy, halogen, sulfhydryl, nitryl, phosphoryl, and sulfonyl;

R₃ is selected from the group consisting of N, CH, and COH; and

R₄ is selected from the group consisting of H, OH, and hydrocarbon.

Exemplary compounds of Formula IV that may be employed in these methods include allopurinol, oxypurinol, and tisopurine.

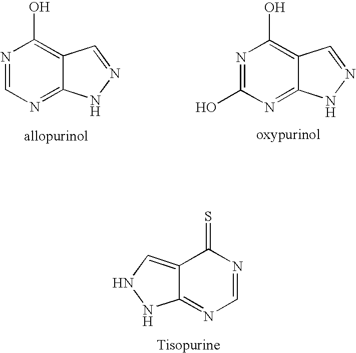

Methodology for the synthesis of pentostatin and related adenosine deaminase inhibitors are described in U.S. Pat. No. 3,923,785.

It will be understood that the exemplary adenosine deaminase and xanthine oxidase inhibitors presented herein are exemplary of a range of inhibitory compounds that may be satisfactorily employed in the present methods. These exemplary compounds are representative of a broader range of suitable compounds that are available in the art and are not intended to be limiting.

Pentostatin (dCF) and coformycin, for example, are naturally-occurring inhibitors of ADA, with Kᵢ values of 2.5×10⁻¹² M (dCF) and 1.0×10⁻¹¹ M (coformycin). Coformycin and dCF are purine analogs that mimic adenosine. The binding between dCF and ADA is effectively irreversible. Thus, dCF is considered a “suicide inhibitor” of ADA. That is, once bound to dCF, the normal functional activity of ADA in converting adenosine to inosine is effectively destroyed. Thus, inhibitors that may be advantageously employed in the present methods for the treatment of myocardial dysfunction bind to ADA or to XO with high affinity, typically with a Kᵢ of between 1.0×10⁻¹⁰ M and 1.0×10⁻¹³ M or between 1.0×10⁻¹¹ M and 1.0×10⁻¹² M.

One or more ADA inhibitor(s) and or one or more XO inhibitor(s) may be administered to a subject in any suitable form for the treatment, prevention, and/or amelioration of myocardial dysfunction that is associated with SIRS and/or sepsis. Examples of suitable formulations for administration include, without limitation, pills, transdermal patches, inhalants, liquids, suppositories, and suspensions. Inhibitory compounds may be administered parenterally (e.g., by injection) and/or through the alimentary tract (e.g., by swallowing). Parenteral administration includes sub-cutaneous, intravenous, intramuscular, and intraarterial injections. Intraarterial and intravenous injections may include administration through a catheter.

ADA and/or XO inhibitors are typically formulated as pharmaceutical compositions such as in sterile injectable preparations, which include sterile injectable aqueous or oleaginous suspensions as are well known to those of skill in the art. The amount of active ingredient that may be combined with a carrier material to produce a single dosage form will vary depending upon the physical characteristics of the patient to be treated, the mode of administration, and the active ingredient used.

The specific dose level for any particular patient will depend on a variety of factors including the activity of the specific inhibitory compound employed; the age, body weight, general health, sex, and diet of the patient; the time and route of administration; the rate of excretion and in vivo metabolism; other drugs which have previously been administered; and the severity of the myocardial dysfunction being treated. All of these factors are well known to those of skill in the art. The administration of one or more inhibitory compound may be at a time and dosage sufficient to achieve substantial improvement in one or more indices of said myocardial dysfunction, as described above, including, for example, left ventricular diastolic function and/or left ventricular systolic function.

All patents, patent application publications, and patent applications, whether U.S. or foreign, and all non-patent publications referred to in this specification are expressly incorporated herein by reference in their entirety.

### EXAMPLES

### Example 1

### Langendorff Rat Isolated Heart Procedure

Male Sprague Dawley, virus-free rats (Harlan) were used in each of the examples presented herein. After arrival and a brief acclimation (72 hours minimum), animals were randomized into control or experimental groups. The control and experimental protocols (Table 1) and experimental group treatments (Table 2) are described below.

TABLE 1
Experimental Group Descriptions

| Group | n | Description |
|---|---|---|
| CON | 8 | Rats that received saline vehicle 1 hr prior to heart |
| | | isolation. Hearts infused with vehicle. |
| LPS | 8 | Rats that received saline vehicle 1 hr prior to heart |
| | | isolation. Hearts infused with LPS |
| | | (10 μg/mL/min) |
| dCF-CON | 6 | Rats that received dCF (1.0 mg/kg, IP) 1 hr prior to |
| | | heart isolation. Hearts infused |
| | | with vehicle. |
| dCF-LPS | 6 | Rats that received dCF (1.0 mg/kg, IP) 1 hr prior to |
| | | heart isolation. Hearts infused with |
| | | LPS (10 μg/mL/min). |
| ALO-CON | 6 | Rats that received ALO (50 mg/kg, IP) 2 hr prior to |
| | | heart isolation. Hearts infused with |
| | | vehicle. |
| ALO-LPS | 6 | Rats that received ALO (50 mg/kg, IP) 2 hr prior to |
| | | heart isolation. Hearts infused with |
| | | LPS (10 μg/mL/min). |

TABLE 2
Summary of Experimental Group Treatment Courses

| Group | dCF | ALO | LPS |
|---|---|---|---|
| CON | −− | −− | −− |
| LPS | −− | −− | + |
| dCF-CON | + | −− | −− |
| dCF-LPS | + | −− | + |
| ALO-CON | −− | + | −− |
| ALO-LPS | −− | + | + |

Rat isolated heart experiments were performed via Langendorff method [85]. As noted in Table 1, rats in the dCF and ALO groups were pre-treated with dCF (1.0 mg/kg IP 1 hour) or ALO (50 mg/kg IP, 2 hours), respectively, prior to removal of the heart. CON hearts received vehicle (IP sterile water). An abdominal midline incision was performed exposing the inferior vena cava into which 1000 units of heparin was administered. Heparin was allowed to circulate for 60 seconds at which time a thoracotomy was performed to expose the heart. The heart was quickly removed, placed in ice cold Krebs-Ringers bicarbonate buffer (KRB, described below) and weighed. The ascending aorta was rapidly cannulated and the heart was attached, via the cannula, to the perfusion apparatus. The hearts were perfused with a modified KRB containing (102 mM NaCl, 4.75 mM KCl, 1.20 mM KH₂PO₄, 1.18 mM MgSO₄, 22.75 mM NaHCO₃, 11.50 mM Glucose, 4.96 mM Na-Pyruvate, 5.40 mM Na-Fumarate and 1 mM CaCl₂), at a temperature of 37.0° C., and pH 7.4 as maintained by vigorous bubbling with 95%/5% O₂/CO₂.

At all times, hearts were perfused with KRB at a pressure of 72-78 mmHg set by the height of the water-jacketed reservoir. The left atrium of the heart was then removed, just above the left atrio-ventricular valve. A latex balloon was used to measure left ventricular pressure as an index of cardiac performance. The balloon was constructed by filling the reservoir tip of a latex condom with degassed water. PE-50 tubing, pre-filled with degassed water and attached to a stopcock/Hamilton screw-top syringe assembly, was flanged and placed into the tip of the condom. Surgical silk (5-0) was used to gather the condom tip around the flanged end of the tubing and form a seal, at which time the excess latex was removed.

The balloon/stopcock assembly was attached to a pressure transducer (Spectramed-Statham) and inserted into the left ventricle to record cardiac performance data via Windaq (DATAQ, CA). Two platinum electrodes, connected to a electrical stimulator (Grass Model 92C), were placed at the lateral edges of the right atria in order to pace the heart at 300 BPM using a voltage 10% greater than that required to capture rhythm. The volume in the latex balloon was increased to produce a left ventricular diastolic pressure (LVdiaP) of 10.0 mmHg.

The heart was then perfused normally for 30 minutes, a period designated as the equilibration phase. During this phase of perfusion hearts had to maintain a LVdiaP of 10.0±2.0 mmHg to be included in the study. At the end of thirty minutes, baseline measurements of coronary flow and left ventricular pressure were made. This time point was designated as 0 minutes. At this time, hearts receiving LPS (10 μg/mL/min) were infused via injection port/stop cock assembly, into the KRB perfusion stream, just above the aortic cannula for a total infusion time of 60 minutes. Hearts not receiving LPS were infused with vehicle (sterile water, 1.25% coronary flow (mL)·min). At 60 minutes, infusion was halted and normal KRB perfusion continued for 90 minutes (total experimental time post-equilibration=150 minutes).

Measurements of coronary flow and left ventricular pressure were taken every 30 minutes starting at 0 minutes. At the termination of the experiment, the left ventricle was rapidly excised from the whole heart, flash frozen in liquid nitrogen and stored at −80.0° C. for further analysis.

Homogenization of Left Ventricular Tissue

A portion of left ventricular tissue previously frozen and stored (approximately 400 mg) was homogenized in ice-cold radio-immunoprecipitation assay (RIPA) buffer containing NaCl 150 mM, EDTA 1 mM, TRIS-HCl 50 mM, Nonidet P-40 1% and Na-deoxycholate 0.25%, pH 7.4. Complete Mini protease inhibitor cocktail (Roche Applied Science, Indianapolis, 11\1) was added to the RIPA buffer prior to homogenization according to manufacturer's specifications and samples were homogenized using a PowerGen 700D homogenizer probe and engine (Fisher Scientific). Tissue samples were homogenized in 3, 10 second bursts at 4° C. Homogenates were aliquotted and stored at −80° C. for further analysis.

Determination of Protein Concentration in Ventricular Homogenates

The protein concentration of left ventricular homogenate samples was determined by bichorionic acid microtitre plate assays (BCA, Pierce). Assays were performed according to the manufacturer's instructions. A protein standard curve was created using dilutions of bovine serum albumin provided with the kit (20-2000 μg/mL) and pippetted, in duplicates, into the microtitre plate. Samples of left ventricular homogenate (20 μL) were pipetted in duplicates into a microtitre plate as well. Working reagent (200 μL) was added to duplicates. The plate was incubated at 37° C. for 1 hour then cooled to room temperature. Absorbance was read at a wavelength of 562 nm using a spectrophotometer (Bio-Tek Instruments, Winooski, Vt.).

Measurement of Left Ventricular Adenosine Deaminase Activity

ADA activity in left ventricular homogenate samples was measured colorimetrically (Berthelot reaction) [86]. Left ventricular homogenate (5 μL) was diluted with 15 μl of red blood cell lysis buffer (RBCLB, 50.0 mM KH₂PO₄ (pH 7.0), 0.125 mM ethylenediamine tetraacetic acid and 0.5 mM MgCl₂) or RBCLB containing 50 uM dCF and loaded in triplicates. Samples that were diluted in RBCLB produced results indicative of total ADA activity. Samples diluted in RBCLB containing 50 μL dCF would yield results indicative of background or non-specific deaminating activity. 80 μl of reaction mixture containing 3.0 mM deoxyadenosine, 0.1 M KH₂PO₄ and 0.5% Triton X 100 (pH 7.0) was added to the triplicate samples and the plated was covered. The enzyme reaction was incubated at 37° C. for 4 hours. The reaction was then stopped by addition of “Solution 1” (53.2 mM Phenol and 1.2 mM Sodium Nitroprusside). Addition of “Solution 2” (1.88 M NaOH and 3.3% Sodium Hypochlorite) initiated the colorimetric reaction which was incubated at 54° C. for 1 hour. Colorimetric reaction was stopped by the addition of 0.5 M NaOH and samples were read at a wavelength of 620 nm via spectrophotometer (Bio-Tek Instruments, Winooski, Vt.).

Statistical Analysis

Statistical analysis was performed on data generated from these experimental protocols using a one-way analysis of variance (one-way ANOVA) with pair wise comparisons (Fisher Least Significant Difference test (LSD)). Data is presented herein as mean±standard error of the mean (SEM).

Animal Characteristics

The age of the rats used in this study was 3-4 months. No significant differences were found in body weight or heart weight among groups (Table 3). Coronary flow did not significantly differ among groups (Table 4).

TABLE 3
Rat Characteristic Data

| | Body Weight (g) | Heart Weight (g) |
|---|---|---|
| Group | (mean ± SEM) | (mean ± SEM) |
| CON | 420 ± 12 | 1.55 ± 0.03 |
| LPS | 456 ± 17 | 1.66 ± 0.09 |
| dCF-CON | 445 ± 07 | 1.58 ± 0.03 |
| dCF-LPS | 423 ± 04 | 1.53 ± 0.03 |
| ALO-CON | 436 ± 04 | 1.49 ± 0.02 |
| ALO-LPS | 445 ± 04 | 1.54 ± 0.02 |

TABLE 4
Coronary Flow Time-course

| | Baseline | 30 min | 60 min | 90 min | 120 min | 150 min |
|---|---|---|---|---|---|---|
| Group | (ml/min) | (ml/min) | (ml/min) | (ml/min) | (ml/min) | (ml/min) |
| CON | 26.3 ± 1.3 | 26.0 ± 1.0 | 25.2 ± 1.1 | 23.9 ± 1.1 | 22.3 ± 1.3 | 27.7 ± 1.3 |
| dCF-CON | 28.1 ± 1.9 | 27.7 ± 1.9 | 26.3 ± 2.1 | 24.5 ± 1.8 | 21.5 ± 1.9 | 19.8 ± 2.0 |
| LPS | 25.0 ± 1.7 | 25.7 ± 1.8 | 25.2 ± 1.5 | 22.9 ± 1.8 | 22.0 ± 1.6 | 19.8 ± 1.7 |
| dCF-LPS | 27.4 ± 1.7 | 28.7 ± 1.2 | 27.8 ± 1.2 | 25.7 ± 1.0 | 22.4 ± 1.1 | 19.6 ± 0.8 |
| ALO-CON | 25.4 ± 2.1 | 25.9 ± 1.8 | 25.1 ± 1.9 | 24.7 ± 1.9 | 24.1 ± 1.6 | 22.6 ± 1.4 |
| ALO-LPS | 24.1 ± 1.3 | 24.2 ± 1.1 | 24.7 ± 1.4 | 24.5 ± 1.4 | 23.4 ± 1.7 | 22.5 ± 1.7 |

Values are expressed as mean ± SEM coronary flow in ml/min
CON and LPS groups n = 8
All other groups n = 6

Among the multiple factors that contribute to the myocardial dysfunction long recognized as a hallmark of sepsis, TNF-α is considered one of the proximal mediators. Increases in myocardial TNF-α mRNA expression and indications of myocardial dysfunction were observed in response to LPS infusion, as discussed above. LPS infusion into rat isolated hearts produced a bi-phasic functional response over time. This response consisted of an initial increase in left ventricular function (highest at 30 minute) followed by a functional decline phase that progressed through 150 minutes.

LPS infusion caused significant cardiac dysfunction, exemplified by decreases in left ventricular systolic (LV developed pressure, +dP/dt) and diastolic (LV diastolic pressure) function, within 150 minutes. Although LPS LV systolic pressure did not significantly differ from CON, further consideration of the individual components of LV systolic pressure (LVdevP and LVdiaP, LVsysP=LVdevP+LVdiaP) suggested the presence of both systolic and diastolic dysfunction in response to LPS infusion.

LPS-induced systolic dysfunction was also exemplified by a significant reduction in LPS LV developed pressure compared to CON, as well as significantly decreased LPS +dP/dt at 150 minutes. When load and heart rate are constant (as they are in this model), +dP/dt is an indicator of myocardial contractility. The significant depression of LPS left ventricular +dP/dt in comparison to its respective baseline value suggests that a significant decrease in contractility contributed to the decrease in both LPS LV systolic pressure (vs. baseline) and LV developed pressure (vs. CON and baseline).

These observations are supported by Braun-Duallaeus and colleagues [88], who reported LV developed pressure and +dP/dt depression in rat isolated hearts in response to LPS. Stamm and colleagues [89] reported a similar response of rat isolated hearts to LPS where LVdevP declined in a time-dependent manner. Additionally, Farias and colleagues [90] reported significant decreases in ventricular systolic performance in septic rats at days 3 and 7 post induction of sepsis in a rat model of sepsis. These investigators reported significant decreases in both LV systolic pressure and +dP/dt which is analogous to our current observations.

The significant increase in LV diastolic pressure in response to LPS indicated reduced diastolic compliance (LV diastolic pressure is an indicator of left ventricular diastolic compliance in this model, with end diastolic volume remaining constant). No significant differences in LPS and CON −dP/dt were observed, which suggested that the rate of relaxation did not contribute to the change in LPS end-diastolic pressure. This also suggested that an additional mechanism contributed to the LPS-induced reduction in diastolic compliance.

The significant increase in LPS LV diastolic pressure described above could be attributed to TNF-α derived affects on capillary permeability and the subsequent progression of edema. If TNF-α induces an increase in overall capillary permeability, then potentially the rate of edema formation would also increase, resulting in decreased diastolic compliance.

These data suggest that an inflammatory challenge (outside of a septic insult) can lead to significant myocardial dysfunction. The model of inflammation described above exhibited responses analogous to those reported for the septic model of Farias and colleagues, in which diastolic dysfunction was exemplified by reduced ventricular compliance [90].

### Example 2

### Real Time PCR Analysis of Gene Expression

Total RNA was isolated from left ventricular tissue using the SV Total Isolation System (Promega Corp., Madison, Wis.). Isolation was performed according to manufacturer instructions and followed by DNAse treatment to eliminate residual genomic DNA contamination. RNA integrity was determined by loading 1.0 μg of total RNA isolate from each sample onto a 1.0% agarose gel and staining with ethidium bromide to visualize the 18 and 28s RNA bands. The amount of RNA recovered was determined using the Ribogreen RNA Quantification Kit (Molecular Probes, Eugene, Oreg.). 1 μg of total RNA was reverse transcribed in a 20-4 volume using random hexamer primers with enzyme and buffers supplied in the cDNA First Strand Synthesis kit (MRI Fermentas, Hanover, Md.). First strand cDNA was treated with ribonuclease H to remove residual RNA.

Real time PCR (qPCR) was performed to measure RNA transcripts for left ventricular TNF-α and ADA1. Three housekeeping genes were selected as follows to produce an intergroup correction factor for target transcripts: Cyclophilin A, hypoxanthine-guanine physphoribosyltransferase (HPRT), and β-actin. Primers were selected using Primer 3 software (Rosen and Skaletsky, Whitehead Institute for Biomedical Research) with the rat genomic sequences for TNF gene, ADA gene, HPRT gene, β-actin gene, and Cyclophilin gene as templates. BLAST (National Center for Biotechnology Information) searches were used to verify that the selected primers were specific for the designated target.

Specific primer sequences, GenBank accession numbers, Sequence Identifiers, and product sizes are disclosed in Table 5. Primers were used in a standard PCR with the 2× Master Mix PCR reagent (MRI Fermentas) and cDNA from the tissue of interest as a template. The thermocycling profile consisted of one cycle of 95° C. for 10 minutes, followed by 41 cycles of 95° C. for 1 minute, 61° C. for 1 minute, and 72° C. for 1 minute and a final cycle of 72° C. for 10 minutes. Products were run on a 1.0% agarose gel and stained with ethidium bromide to confirm that only one band was amplified and that no primer dimers were present. PCR products were then column-purified (QIAGEN, Valencia, Calif.) and sequenced to confirm target specificity. For real-time PCRs, SYBR PCR Master Mix (Bio-Rad Laboratories, Hercules, Calif.) was used, and thermocycling and fluorescence detection was performed using a Stratagene Mx3000p real-time PCR machine.

TABLE 5
Real Time PCR Primer Characteristics

| Gene | Sequence | Forward Sequence | Product Size |
|---|---|---|---|
| (Accession #) | Identifier | Reverse Sequence | (bp) |
| TNF-α | SEQ ID NO: 1 | 5′-CAAATGGGCTCCCTCTCATC-3′ | 117 |
| (X66539) | SEQ ID NO: 2 | 5′-GCTCCTCTGCTTGGTGGTTT-3′ | |
| ADA1 | SEQ ID NO: 3 | 5′-AAGGTCCGGTCCATCTTGTG-3′ | 183 |
| (AB059655) | SEQ ID NO: 4 | 5′-ATCCTTCACTGCACCCTCGT-3′ | |
| B-actin | SEQ ID NO: 5 | 5′-CTGGGACGATATGGAGAAGA-3′ | 205 |
| (NM_031144) | SEQ ID NO: 6 | 5′-ACCAGAGGCATACAGGGACA -3′ | |
| Cyclophilin A | SEQ ID NO: 7 | 5′-TGGTCTTTGGGAAGGTGAAAG-3′ | 109 |
| (NM_008907) | SEQ ID NO: 8 | 5′-TGTCCACAGTCGGAAATGGT-3′ | |
| HPRT | SEQ ID NO: 9 | 5′-CAGTCAACGGGGGACATAAA-3′ | 183 |
| (NM_012583) | SEQ ID NO: 10 | 5′-AGAGGTCCTTTTCACCAGCAA-3′ | |

After confirmation of primer efficiency and specificity, the concentrations of purified products generated by standard PCR were determined using Molecular Probe's Picogreen DNA quantification kit, and PCR products were serially diluted to obtain standards containing 10¹, 10², 10³, 10⁴, 10⁵, and 10⁶ copies of synthetic template. Standards were then amplified by real-time PCR, and standard curves were generated using Stratagene Mx3000P software. The slope of a standard curve for each template examined was approximately 1, indicating that the efficiency of amplification was 100%, meaning that all templates in each cycle were copied. Standard curves were constructed for all transcripts examined. In addition, total RNA samples that were not reverse-transcribed and a no-DNA control were run on each plate to control for genomic DNA contamination and to monitor potential exogenous contamination, respectively.

### Example 3

### Treatment of Myocardial Dysfunction with the Adenosine Deaminase (ADA) Inhibitor Pentastatin (dCF)

This Example discloses that Pentastatin (dCF) is effective in the treatment of myocardial dysfunction associated with SIRS and sepsis.

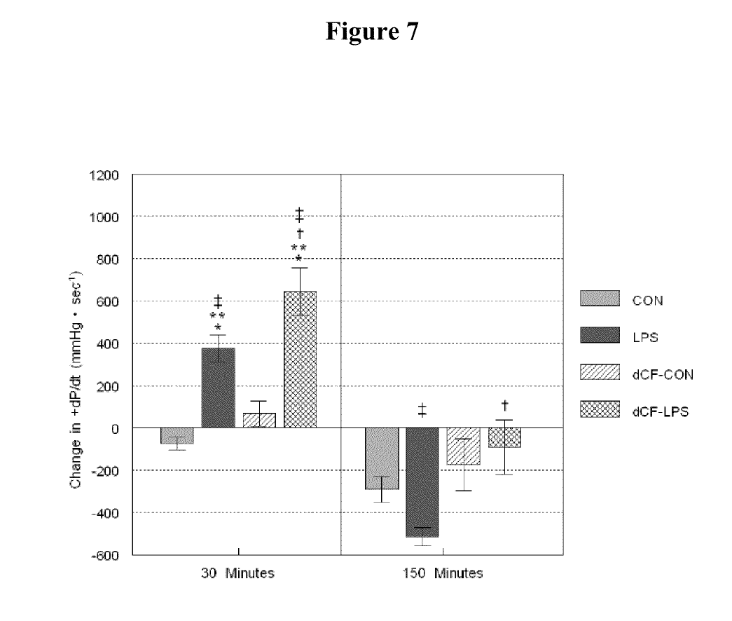

The influence of Pentostatin (dCF) on cardiac dysfunction was investigated using the model system described in Example 1 (FIG. 7). dCF inhibition of ADA prior to an LPS-induced inflammatory challenge was examined to determine the effect of this inhibition on LPS-induced cardiac dysfunction and TNF-α and/or ADA1 mRNA expression. The following control and experimental groups were used: CON, LPS, dCF-CON and dCF-LPS (Table 1).

Left Ventricular (LV) Function

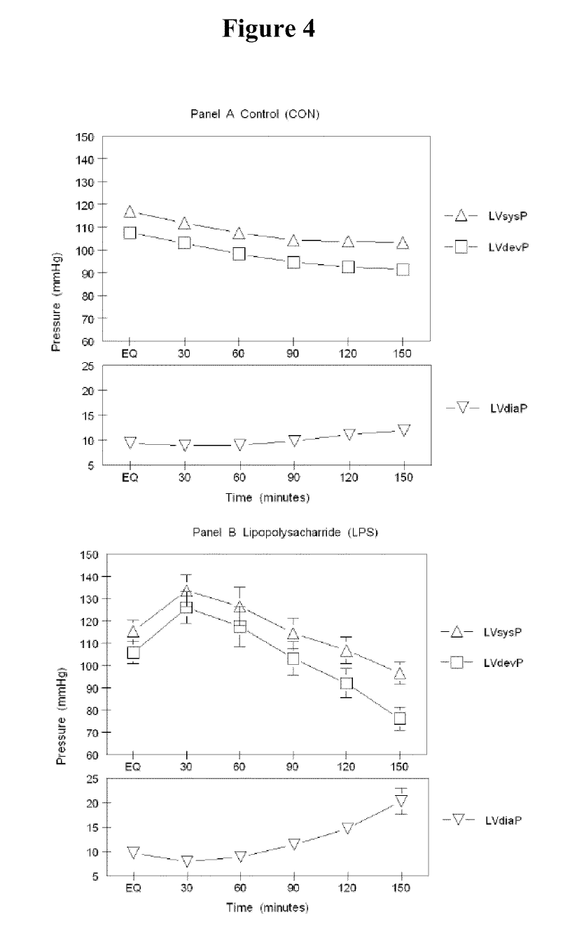

Steady decreases in LV systolic pressure and LV diastolic pressure over time were observed in the vehicle control group (CON); CON LV diastolic pressure increased steadily over time (FIG. 4A). Infusion of LPS resulted in a biphasic pattern in LV systolic pressure, LV developed pressure, and LV diastolic pressure (FIG. 4B). The initial phase was characterized by a steep rise in LV systolic pressure and LV developed pressure followed by a decline phase that continued to the end of the time course.

An opposite effect was observed for LPS LV diastolic pressure, which consisted of an initial decrease in LV diastolic pressure followed by an increase. The changes in left ventricular functional parameters during these two phases were, therefore, analyzed separately. Functional increases peaked at 30 minutes. The greatest decline in function was observed at the end of the experiment (150 minutes). Thus, functional parameter comparisons were made at 30 and 150 minutes. This biphasic response was observed in all indices of left ventricular function.

The Fisher LSD was used to determine whether 30 and 150 min values differed from baseline values. In addition, changes from baseline values (e.g., 30 or 150 minute value−respective baseline value) were compared among groups.

Left Ventricular Systolic Function

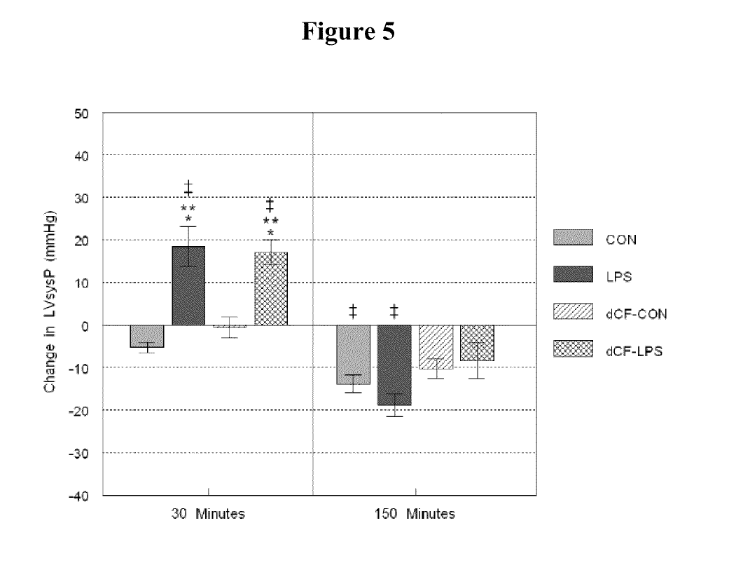

LPS and dCF-LPS LV systolic pressure was significantly greater at 30 minutes as compared to respective baseline values (FIG. 5). At 30 minutes, the increase in LPS and dCF-LPS LV systolic pressure was similar between these groups (18.4±1.2 mmHg and 17.1±3.0 mmHg respectively), but significantly greater than the CON and dCF-CON groups. At 150 minutes, both CON and LPS LV systolic pressure was significantly decreased compared to respective baseline values. However, no significant differences in LV systolic pressure were found among groups.

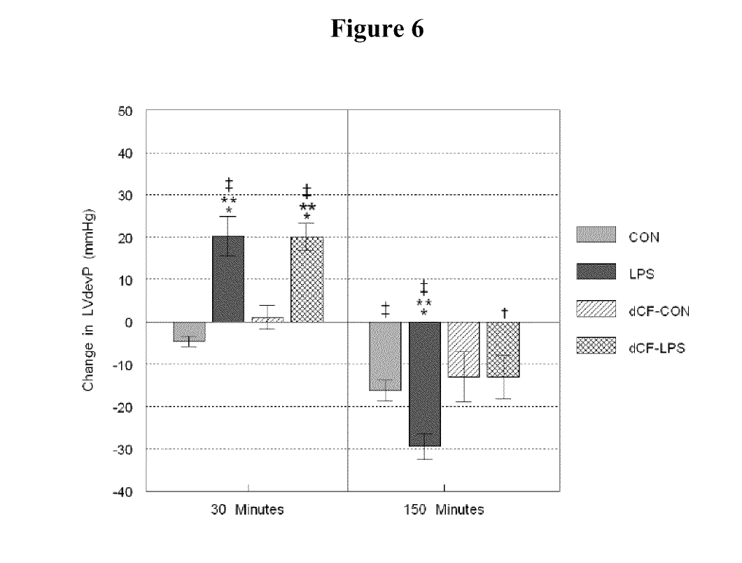

LV developed pressure was also measured in all hearts (LV developed pressure=LV systolic pressure−LV diastolic pressure) (FIG. 6). At 30 minutes, LPS and dCF-LPS LV developed pressure was significantly increased compared to respective baseline values. The increase in LV developed pressure was similar between the LPS and dCF-LPS groups (20.2±4.8 and 20.1±3.2 mmHg respectively), but significantly greater compared to both the CON and dCF-CON groups. At 150 minutes, CON and LPS LV developed pressure was significantly decreased compared to respective baseline values, while dCF-CON and dCF-LPS LV developed pressure were not significantly different from their respective baseline values. At 150 minutes, LPS LV developed pressure was significantly decreased compared to the CON, dCF-CON and dCF-LPS groups. No differences in LV developed pressure were found among the three latter groups.

The rate of left ventricular pressure generation (+dP/dt) was calculated from left ventricular pressure data acquired via WINDAQ (FIG. 7). It is the first derivative of left ventricular pressure over time. At 30 minutes, the increases in LPS and dCF-LPS +dP/dt were significantly greater compared to respective baseline values. The LPS increase in +dP/dt (644±111) was greater than the dCF-LPS increase in +dP/dt (376±65). However, both the LPS and dCF-LP increases were significantly greater compared to the CON and dCF-CON +dP/dt values. At 150 minutes, only the increase in the LPS +dP/dt was significantly lower than its baseline value, and this value was also significantly lower compared to the dCF-LPS +dP/dt. No differences were found in +dP/dt among the CON, dCF-CON and dCF-LPS.

Left Ventricular Diastolic Function

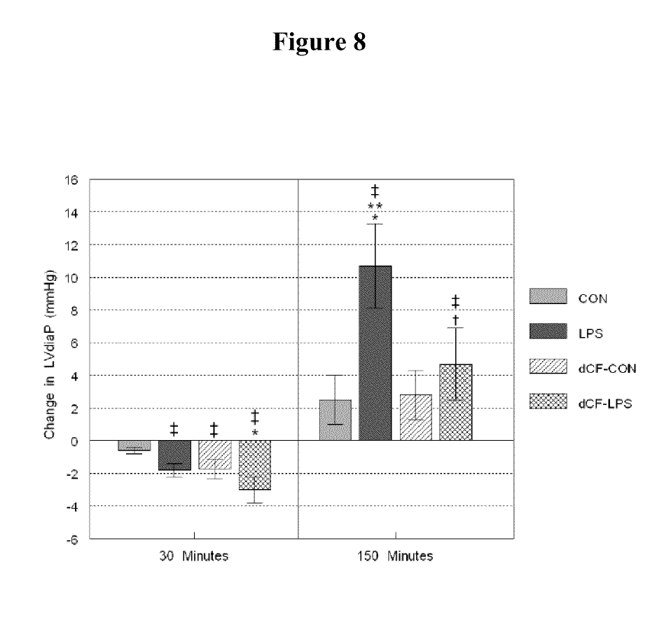

LPS, dCF-CON, and dCF-LPS LV diastolic pressure was significantly lower at 30 minutes as compared to respective baseline values (FIG. 8). No change was found in the CON LV diastolic pressure compared to baseline. LV diastolic pressure was similar among the CON, LPS and dCF-CON groups. However, the decrease in dCF-LPS LV diastolic pressure was greater compared to the CON group. At 150 minutes, both LPS and dCF-LPS group LV diastolic pressure were significantly elevated compared to respective baselines. LPS LV diastolic pressure was significantly elevated compared to all groups.

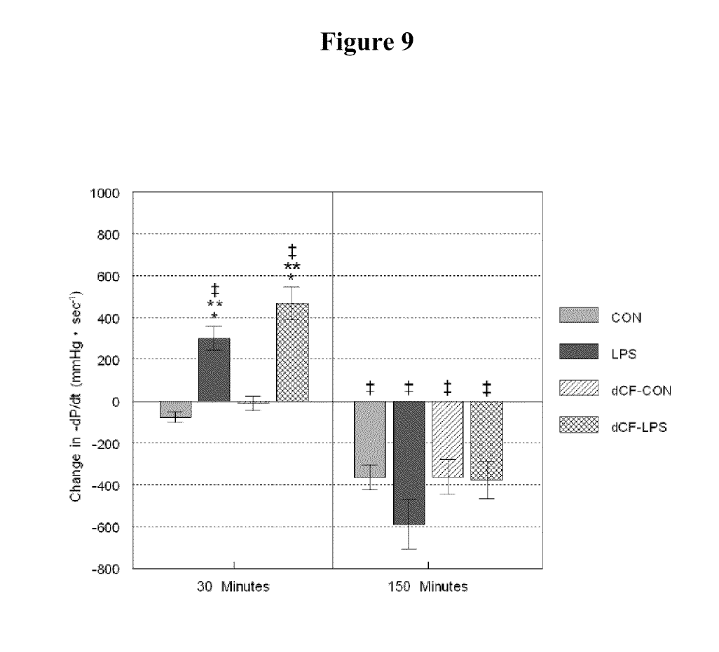

The rate of ventricular relaxation (−dP/dt) was calculated in the same manner as +dP/dt (FIG. 9). At 30 minutes, the LPS and dCF-LPS −dP/dt were significantly greater compared to respective baseline values, while no differences were found in CON and dCF-CON DC100−dP/dt values compared to baseline values. The increases in LPS and dCF-LPS −dP/dt were similar, but significantly greater compared to the CON and dCF-CON groups. At 150 minutes, −dP/dt was significantly lower in all groups compared to respective baselines. No significant differences in −dP/dt were found among groups.

Left Ventricular Adenosine Deaminase Activity

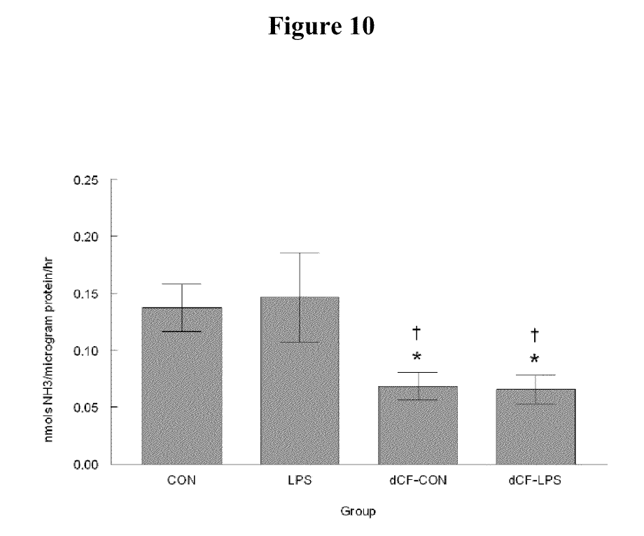

Adenosine deaminase activity in LV tissue homogenates was measured only at the 150 minute time point (FIG. 10). No significant differences were found in LV ADA activity between CON and LPS hearts. However, a significant decrease was found in dCF-CON and dCF-LPS LV ADA activity compared to the CON and LPS hearts.

Housekeeping Gene Expression

A housekeeping gene was selected for normalization of the real-time PCR data for the TNF-α and ADA1 transcripts. Three potential gene (mRNA) products were analyzed for this purpose: CYC, β-actin and HPRT. Neither CYC nor β-actin copy numbers were found to differ significantly between groups. HPRT copy numbers demonstrated significant inter- and intra-group variability, making it an unreliable candidate as a housekeeping gene. β-actin was selected for use as the normalizing housekeeping gene due to its compatibility with the above-described experimental groups. Copy number data for these three gene products is shown in Table 6.

TABLE 6
Housekeeping Gene Copy Number

| | CYC | HPRT | β-actin |
|---|---|---|---|
| CON | 3374 ± 230 | 26,591 ± 1,423 | 5,305 ± 375 |
| dCF-CON | 3956 ± 392 | 18,786 ± 7,494 | 7,470 ± 437 |
| LPS | 3,623 ± 380 | 38,368 ± 4,784 | 5,831 ± 592 |
| dCF-LPS | 3,657 ± 380 | 47,617 ± 8,619*# | 6,062 ± 988 |

*signifies a statistically significant difference from CON
#signifies a statistically significant difference from dCF-CON

Left Ventricular mRNA Expression

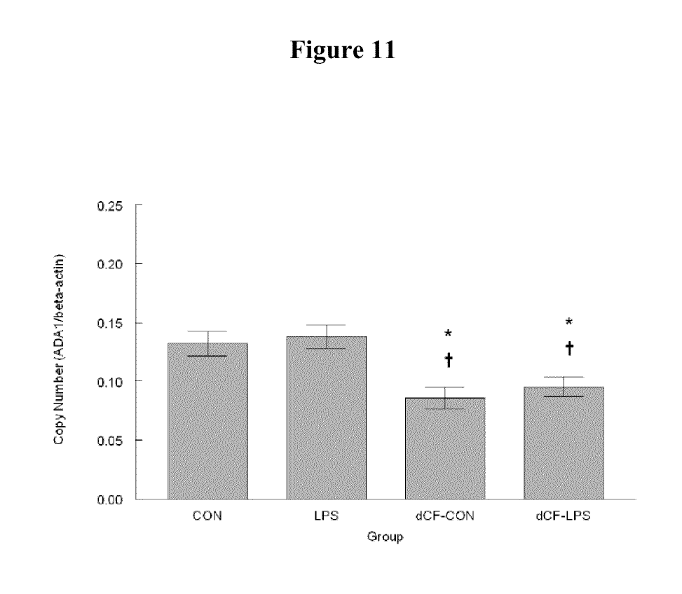

As shown in FIG. 11, left ventricular dCF-CON and dCF-LPS ADA1 copy numbers were significantly lower as compared to the CON and LPS hearts.

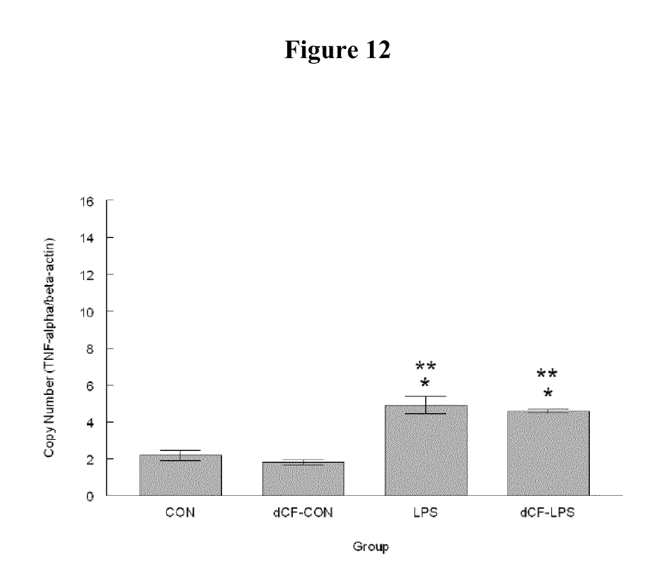

TNF-α mRNA expression increased significantly in response to LPS infusion as compared to the CON and dCF-CON groups (FIG. 12). Treatment with dCF did not significantly alter TNF-α mRNA expression as compared to LPS hearts. No difference was found in LPS-induced increases in TNF-α mRNA between the LPS (alone) and dCF-LPS hearts.

Conclusions

These data demonstrate that ADA inhibition protects systolic and diastolic cardiac function after LPS-infusion but does not affect LPS-induced myocardial TNF-α mRNA expression. Therefore, these data do not support a connection between myocardial functional effects and changes in myocardial TNF-α. In contrast, previous reports indicated that dCF inhibition of ADA resulted in a concomitant decrease in tissue TNF-α protein in a model of rat acute peritonitis [82]. Thus, surprisingly, it appears that myocardial ADA may affect LPS-induced cardiac dysfunction by some other mechanism.

Inhibition of ADA by dCF did not affect the initial increased functional response to LPS-infusion. ADA inhibition did, however, prevent the magnitude of decline in systolic and diastolic cardiac functional parameters seen in LPS hearts at the end of the time course. This was exemplified by improved 150 minute values, similar to that of CON hearts, for LV developed pressure, +/−dP/dt and LV diastolic pressure. The effect of ADA inhibition by dCF on cardiac function is supported by previous work in models of ischemia-reperfusion and myocardial stunning where inhibition of ADA activity resulted in improved function after a cardiac event that results in dysfunction [27, 79-81]. Specifically, Bolling and colleagues [79] reported a significant improvement in LVdevP in response to dCF administration during the reperfusion phase of an ischemia-reperfusion protocol in rabbit isolated hearts.

Myocardial ADA activity was not elevated by LPS infusion in this model. However, even in the absence of a significant LPS-induced elevation in ADA, ADA activity was significantly inhibited with dCF pre-treatment.

It was surprising that ADA inhibition by dCF did not affect TNF-α mRNA expression after LPS infusion. This finding is in contrast to previously published findings where dCF-inhibition of ADA activity during a septic insult, resulted in an attenuation of TNF-α production [27]. Significant elevations of ADA activity measured in previous experiments were observed initially at 24 hours after initiating sepsis through a polymicrobial challenge. Concomitant with dCF-inhibition of ADA activity, a significant reduction of ADA1 mRNA expression was observed. While there is currently little data to support or refute this observation, Law and colleagues [82] reported a sustained inhibition of ADA activity over a period of days in a rat model of sepsis. Thus, dCF may affect transcriptional regulators for ADA1 mRNA expression.

In summary, ADA activity contributes to cardiac dysfunction in an LPS-induced model of inflammation. It does not, however, regulate cardiac TNF-α mRNA expression in the initial 150 minutes after LPS administration. Inhibition of myocardial ADA activity by dCF during LPS-stimulation was accompanied by a reduction in ADA1 mRNA expression.

Two independent mechanisms involving sphingosine and NO may play a role in this observed LPS-induced myocardial dysfunction. Oral and colleagues [62] suggested a mechanism by which the immediate negative inotropic effects of TNF-α are mediated via membrane bound sphingomyelinase and TNFR1. These investigators reported a significant increase in free sphingosine production via sphingomyelinase in response to exogenous TNF-α which resulted in a significant reduction in the amplitude of shortening of isolated cardiac myocytes. In related experiments, the investigators observed significantly reduced myocyte shortening when exogenous sphingosine was added in conjunction with TNF-α.

In addition to the reported actions of sphingosine, an NO-dependent mechanism for TNF-α mediated myocardial depression has also been proposed [5]. Inducible nitric oxide synthase levels may become elevated in response to TNF-α, resulting in increased NO production. NO may act to desensitize the cardiac myofilaments to calcium, resulting in a sustained contractile dysfunction [63, 64]. Together or independently, these mechanisms may contribute to the observed myocardial systolic dysfunction observed in this study.

### Example 4

### Treatment of Myocardial Dysfunction with the Xanthine Oxidase (XO) Inhibitor Allopurinol (ALO)

This Example discloses that Allopurinol (ALO) is effective in the treatment of myocardial dysfunction associated with SIRS and sepsis.

The influence of xanthine oxidase (XO) on cardiac dysfunction was investigated using the model system described in Example 1. Allopurinol (ALO) inhibition of XO prior to an LPS-induced inflammatory challenge was examined to determine the effect of this inhibition on LPS-induced cardiac dysfunction and TNF-α and/or ADA1 mRNA expression. The following control and experimental groups were used: CON, LPS, ALO-CON and ALO-LPS (Table 1).

Left Ventricular Systolic Function

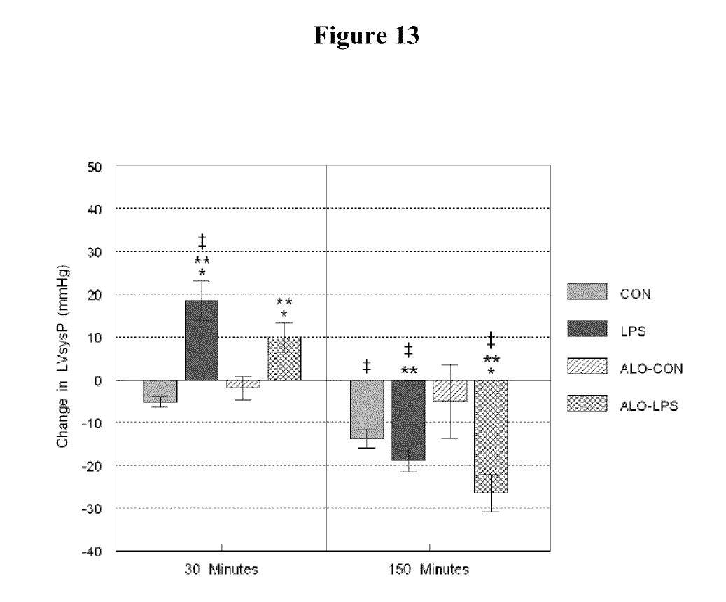

At 30 minutes only the LPS LV systolic pressure value was significantly greater as compared to its respective baseline (FIG. 13). The increases in LPS and ALO-LPS LVsysP were of a similar magnitude, but significantly greater compared to both control groups. At 150 minutes, CON, LPS, and ALO-LPS LVsysP were significantly decreased as compared to respective baseline values (no change was found in the ALO-CON group compared to baseline). Decreases in LPS and ALO-LPS LV systolic pressures were of a similar magnitude. However, the decrease in ALO-LPS was significantly lower as compared to the CON and ALO-CON.

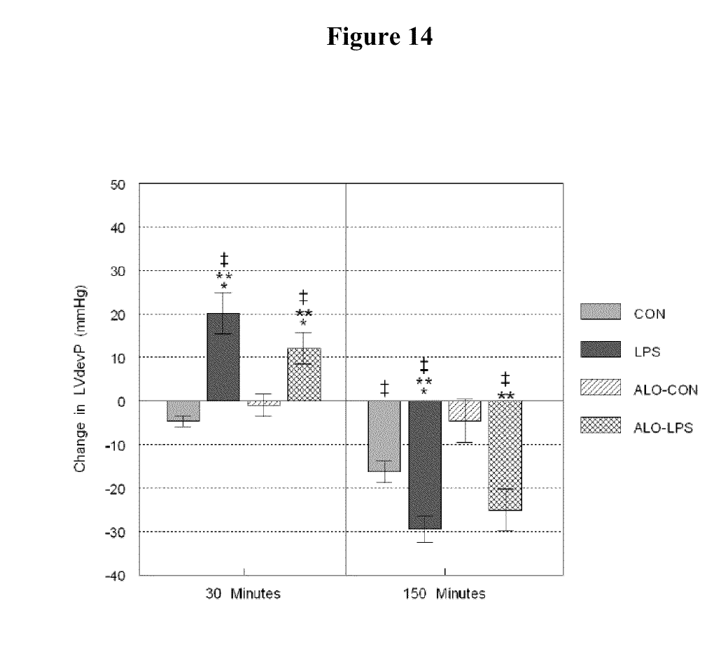

At 30 minutes, LPS and ALO-LPS LV developed pressure was significantly greater as compared to respective baseline values (FIG. 14). Increases in LPS and ALO-LPS LV developed pressure were of a similar magnitude, but significantly greater as compared to both control groups. At 150 minutes, CON, LPS and ALO-LPS LV developed pressures were significantly decreased as compared to respective baseline. No changes were found in ALO-CON as compared to baseline. Decreases in LPS and ALO-LPS LV developed pressures were of a similar magnitude and significantly lower as compared to the ALO-CON.

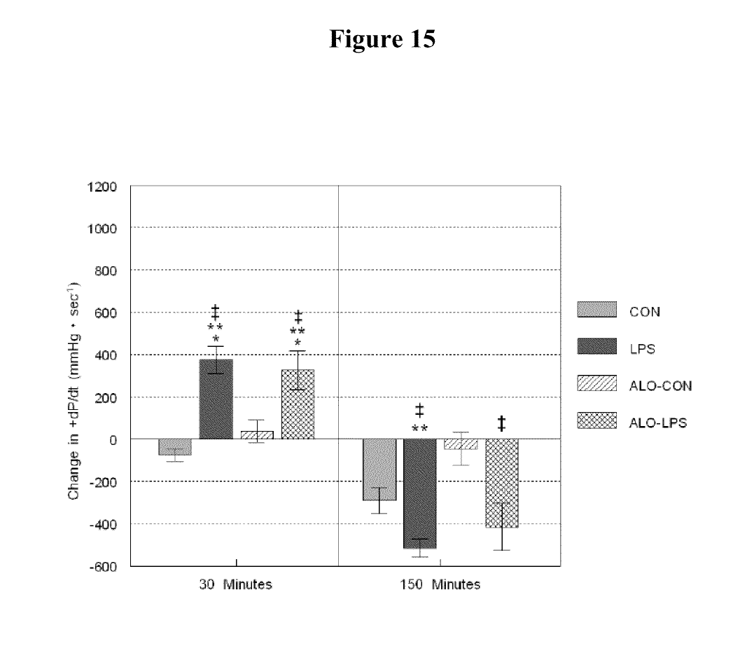

At 30 minutes, LPS and ALO-LPS +dP/dt was significantly greater as compared to respective baseline values, while no differences were found in the CON and dCF-CON +dP/dt as compared to their respective baseline values (FIG. 15). Increases in LPS and ALO-LPS +dP/dt were of a similar magnitude, but significantly greater as compared to the CON and ALO-CON groups. At 150 minutes, LPS and ALO-LPS +dP/dt were significantly decreased compared to respective baseline values, while control groups +dP/dt remained unchanged.

Left Ventricular Diastolic Function

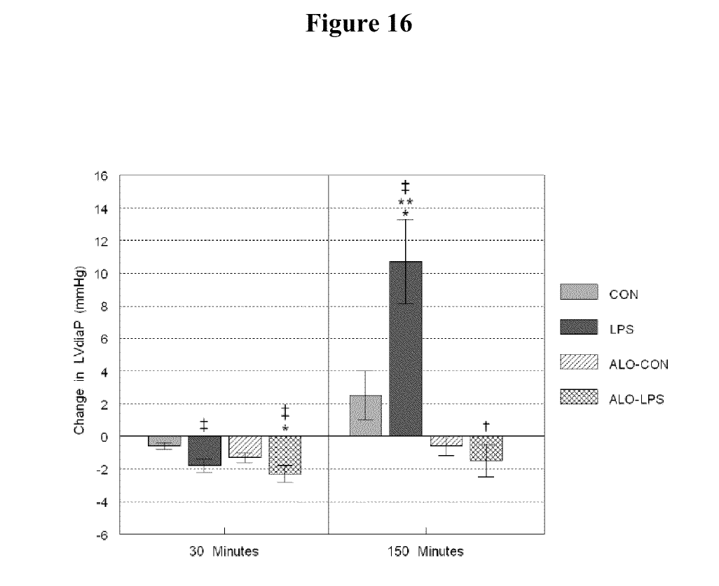

At 30 minutes LPS and ALO-LPS LV diastolic pressure were significantly decreased compared to respective baseline values (FIG. 16). No changes were found in the CON and ALO-CON groups compared to their respective baseline values. No differences in LV diastolic pressure were found among the CON, LPS and ALO-CON groups. However the decrease in ALO-LPS diastolic pressure was significantly greater compared to the CON group. At 150 minutes, LPS LV diastolic pressure was significantly greater as compared to its respective baseline value, while CON, ALO-CON and dCF-LPS LV diastolic pressures remained unchanged. LPS LV diastolic pressure was significantly greater as compared to CON, ALO-CON and ALO-LPS LV diastolic pressure values.

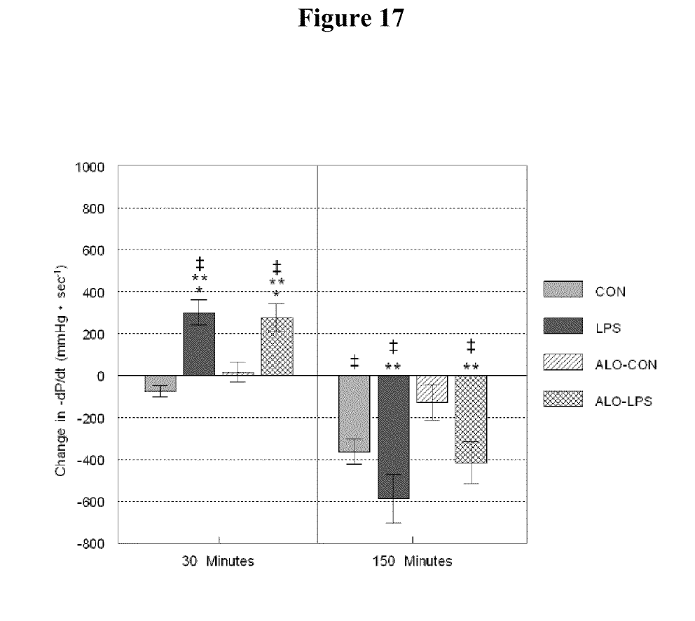

At 30 minutes the value changes in both LPS and ALO-LPS −dP/dt were significantly increased as compared to both control groups (CON and ALO-CON) (FIG. 17). The magnitude of the −dP/dt increase was similar between LPS and ALO-LPS hearts. At 150 minutes, LPS and ALO-LPS −dP/dt were significantly lower than ALO-CON but not CON −dP/dt. CON, LPS and ALO-LPS −dP/dt were significantly decreased from respective baseline values while ALO-CON −dP/dt remained unchanged.

Left Ventricular Adenosine Deaminase Activity

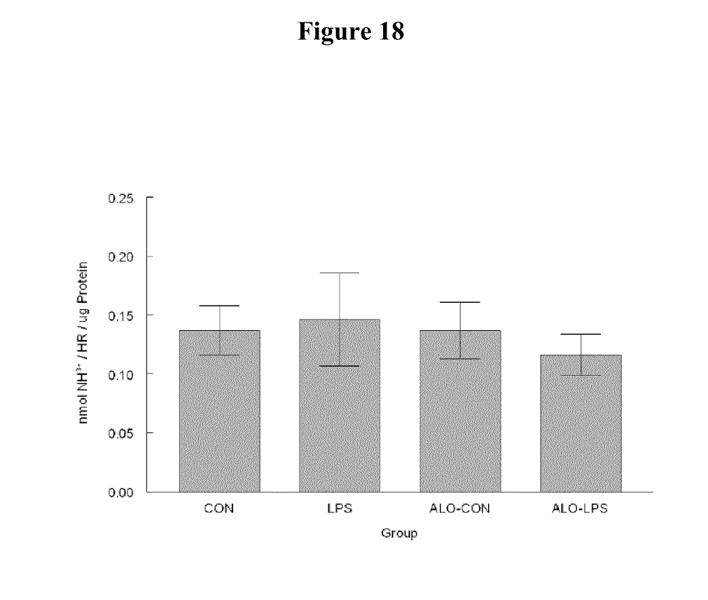

Adenosine deaminase activity was measured colorimetrically. There were no statistically significant differences found among the groups (FIG. 18).

Left Ventricular mRNA Expression

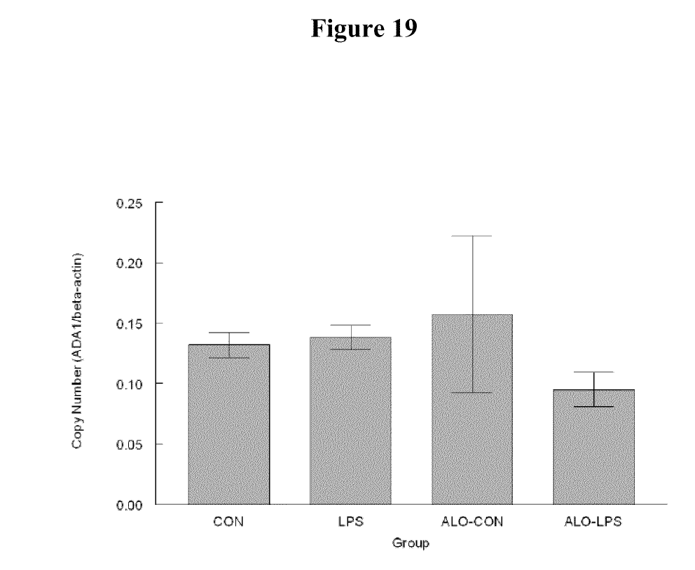

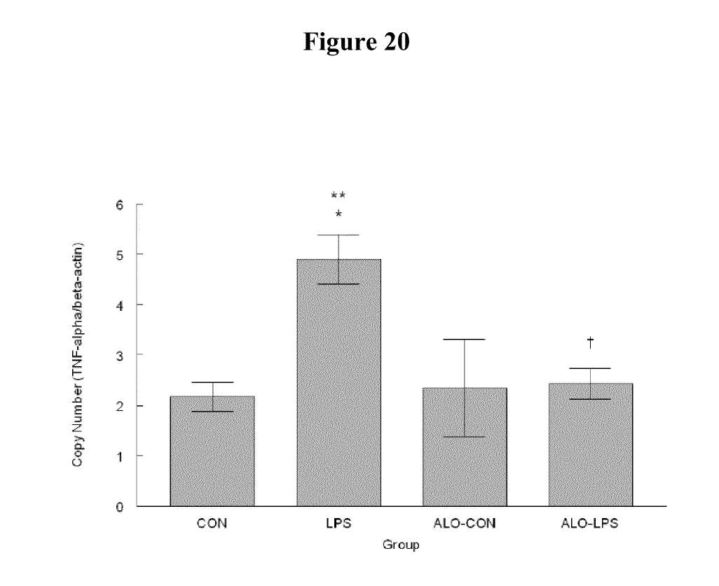

No significant differences in ADA1 mRNA expression were found among groups (FIG. 19). LPS TNF-α mRNA expression was greater as compared to the CON and ALO-CON groups. No changes were found in ALO-LPS TNF-α mRNA expression. ALO-LPS TNF-α mRNA expression was significantly lower than LPS (FIG. 20).

Conclusions

These data demonstrate that endogenous XO inhibition protects diastolic (but not systolic) cardiac function after LPS-infusion and also inhibits TNF-α mRNA expression. In contrast to dCF, administration of ALO (XO inhibitor) significantly reduced TNF-α mRNA expression in response to LPS in comparison to LPS infusion alone. These findings are supported by previous studies where exogenous XO elevated TNF-α production in the heart. In these studies, exogenous XO combined with exogenous substrate (HX) induced cardiac TNF-α production. Meldrum and colleagues [26] used an HX/XO system to promote ROS production in a rat isolated heart model. There they observed a significant increase in myocardial TNF-α production.

In the above-described model, inhibition of XO by ALO resulted in the attenuation of TNF-α mRNA expression after LPS infusion. The mechanism by which endogenous XO contributes to cardiac TNF-α mRNA expression could reasonably be attributed to ROS generation. When XO metabolizes HX and xanthine, ROS are produced in the form of hydrogen peroxide. ROS signaling via the transcription factor NF-κB has been shown to promote TNF-α production [76]. In our model, the inhibition of XO by ALO would have resulted in a reduction in ROS byproduct formation. This would potentially contribute to the reduced cardiac TNF-α mRNA expression observed here.

Inhibition of XO by ALO also affected cardiac function during LPS-infusion. ALO inhibition of XO did not statistically change the systolic response to LPS compared to LPS infusion alone as exemplified by LPS comparable 150 minute values for LV systolic and developed pressures and +dP/dt. On the other hand, ALO inhibition of XO resulted in an attenuation of the LPS-induced increase in LV diastolic pressure at 150 minutes similar to dCF. ALO inhibition did not significantly affect −dP/dt in comparison to LPS alone.

This suggests that endogenous XO activity, and most likely the ROS produced by XO, contributes to LPS-induced diastolic dysfunction by decreasing ventricular compliance but not the rate of ventricular relaxation. While the effect of XO inhibition on myocardial TNF-α mRNA expression can be attributed to ROS generation and subsequent actions on TNF-α regulating transcription factors like NF-κB [26, 66, 93], its effects on cardiac function are not as easily explained.

ALO inhibition of XO activity has been shown to improve cardiac systolic function in both human and an animal model of heart failure [94, 95]. Inhibition of XO by ALO resulted in improvements in the left ventricular ejection fraction of humans in heart failure as demonstrated by Cingolani and colleagues [94]. The data presented by Cingolani and colleagues contrast our finding where XO inhibition by ALO did not improve ventricular systolic function. This was exemplified by significant decreases in LVsysP, LVdevP and +dP/dt in ALO-LPS hearts which were all comparable to decreases found in LPS hearts.

In a dog model of heart failure, inhibition of XO by ALO had a positive inotropic effect combined with decreased myocardial oxygen consumption and increased myofilament calcium sensitivity as shown by Ekelund and colleagues [95]. While these investigators' data also contrast with the functional effects of XO inhibition presented herein, they may provide some insight.

A reduction in myocardial oxygen demand resulting from the inhibition of XO activity might indirectly contribute to the attenuation of LPS-induced diastolic dysfunction (compliance). If the heart is able to maintain a steady aerobic production of high energy phosphate molecules (ATP) then diastolic dysfunction would not be present as it would be in a state where ATP was not readily available, as in myocardial ischemia or coronary hypoperfusion. Additionally we could postulate that since the inhibition of endogenous XO activity resulted in a significant reduction in LPS-induced TNF-α mRNA expression, protein production (although not measured in this study) was also significantly reduced. Reduced TNF-α protein production would limit TNF-α actions on capillary permeability. This would most likely reduce the rate by which LPS-induced edema would occur, therefore preventing decreases in ventricular diastolic compliance.

Endogenous XO activity may play a role in the modulation of cardiac TNF-α mRNA expression in response to LPS. In addition, XO activity may be modulating LPS-induced cardiac dysfunction, though this role is not yet clear.

In summary, while cardiac TNF-α does not appear to be modulated by ADA activity, ADA may play a role in cardiac dysfunction in the context of an inflammatory insult. In addition, endogenous XO activity may play a role in the modulation of LPS-stimulated cardiac TNF-α mRNA expression. Adenosine metabolism is directly relevant to both the modulation of cardiac dysfunction by ADA and the modulation of TNF-α mRNA expression by XO activity.

Thus, the above data indicate a potential use for ADA inhibitors to ameliorate SIRS- and sepsis-related myocardial dysfunction. In particular, inhibitors of ADA-2 (e.g. pentostatin) may be administered to ameliorate myocardial dysfunction associated with those conditions.

What is claimed is:

1. A method for the treatment of myocardial dysfunction associated with SIRS and/or sepsis in a patient, said method comprising the step of administering to said patient a composition comprising an adenosine deaminase (ADA) inhibitor at a time and dosage sufficient to achieve substantial improvement in one or more indicia of myocardial dysfunction.

2. The method of claim 1 wherein said indicia of myocardial dysfunction is selected from the group consisting of left ventricular (LV) systolic pressure; LV diastolic pressure; and rates of ventricular pressure generation or relaxation, cardiac output, or left ventricular ejection fraction.

3. The method of claim 1 wherein said adenosine deaminase inhibitor is an ADA-2 inhibitor.

4. The method of claim 1 wherein said ADA inhibitor is a compound of Formula I: 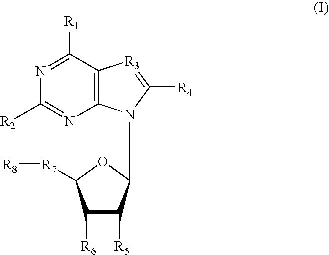
wherein
R₁ and R₂ are independently selected from the group consisting of H, OH, NH₂, OCH₃, CH₃, amino, amide, alkyl, alkoxyl, sulfhydryl, alkylthio, halogen, nitryl, phosphoryl, sulfinyl, and sulfonyl;
R₃ is selected from the group consisting of CH, N, or an acyclic substituent,
R₄ is selected from the group consisting of H, OH, halogen, alkyl, alkoxyl, amino, amide, sulfhydryl, nitryl, phosphoryl, sulfinyl, and sulfonyl;
R₅ is selected from the group consisting of H, OH, and halogen;
R₆ is selected from the group consisting of H, OH, and halogen;
R₇ is selected from the group consisting of CH₂ and phosphoryl; and
R₈ is selected from the group consisting of H, OH, amino, alkoxy, alkyl, and phosphoryl.

5. The method of claim 1 wherein said ADA inhibitor is a compound of Formula II: 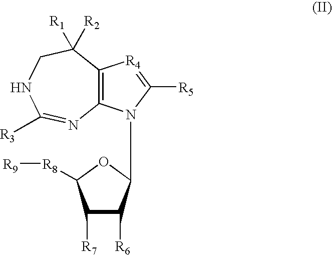
wherein
R₁, R₂, R₃, and R₅ are each independently selected from the group consisting of H, OH, amino, amide, alkyl, alkylthio, alkoxy, halogen, sulfhydryl, nitryl, phosphoryl, and sulfonyl;
R₄ is selected from the group consisting of CH, N, and an acyclic substituent such as CH—O—CH(COOH)₂;
R₆ is selected from the group consisting of halogen, H, and OH;
R₇ is selected from the group consisting of halogen, H, and OH;
R₈ is selected from the group consisting of CH₂ and phosphoryl; and
R₉ is selected from the group consisting of H, OH, amino, alkoxy, alkyl, and phosphoryl.

6. The method of claim 5 wherein said ADA inhibitor is 2′-deoxy-8-epi-2′-fluorocoformycin:

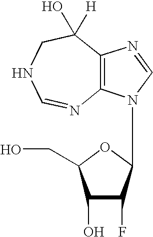

7. The method of claim 1 wherein said ADA inhibitor is coformycin:

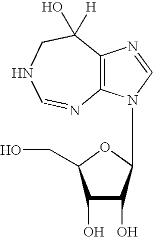

8. The method of claim 1 wherein said ADA inhibitor is 2′-deoxycoformycin (pentostatin):

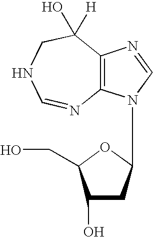

9. The method of claim 1 wherein said ADA inhibitor is 2-chloropentostatin:

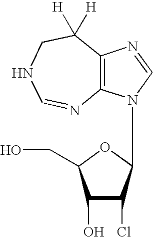

10. The method of claim 1 wherein said ADA inhibitor is a compound of Formula III: 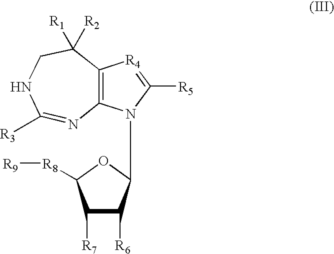
wherein
R₁, R₂, R₃, and R₅ are each independently selected from the group consisting of H, OH, amino, amide, alkyl, alkylthio, alkoxy, halogen, sulfhydryl, nitryl, phosphoryl, and sulfonyl;
R₄ is selected from the group consisting of CH, N, and an acyclic substituent such as CH—O—CH(COOH)₂;
R₆ is selected from the group consisting of halogen, H, and OH;
R₇ is selected from the group consisting of halogen, H, and OH;
R₈ is selected from the group consisting of CH₂ and phosphoryl; and
R₉ is selected from the group consisting of H, OH, amino, alkoxy, alkyl, and phosphoryl.

11. The method of claim 1 wherein said ADA inhibitor is isocoformycin:

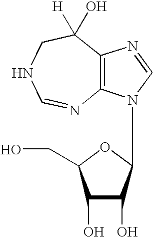

12. A method for the treatment of myocardial dysfunction associated with SIRS and/or sepsis in a patient, said method comprising the step of administering to said patient, a composition comprising a xanthine oxidase (XO) inhibitor at a time and dosage sufficient to achieve substantial improvement in one or more indicia of myocardial dysfunction.

13. The method of claim 12 wherein said indicia of myocardial dysfunction is selected from the group consisting of left ventricular (LV) systolic pressure and LV diastolic pressure; LV developed pressure; and rates of left ventricular pressure generation or relaxation, cardiac output, or left ventricular ejection fraction.

14. The method of claim 12 wherein said XO inhibitor is a compound of Formula IV: 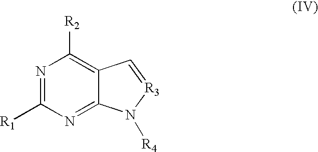
wherein
R₁ and R₂ are each independently selected from the group consisting of H, OH, O, S, halogen, mercapto, cyano, methylamine, hydrocarbon, amino, amide, alkyl, alkylthio, alkoxy, halogen, sulfhydryl, nitryl, phosphoryl, and sulfonyl;
R₃ is selected from the group consisting of N, CH, and COH; and
R₄ is selected from the group consisting of H, OH, and hydrocarbon.

15. The method of claim 14 wherein said compound of Formula IV is a purine analog selected from the group consisting of allopurinol, oxypurinol, tisopurine.

16. The method of claim 15 wherein said purine analog is allopurinol:

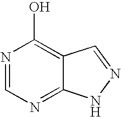

17. The method of claim 13 wherein said purine analog is oxypurinol:

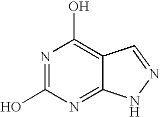

18. The method of claim 13 wherein said purine analog is tisopurine:

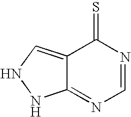

19. A composition for the treatment of myocardial dysfunction, comprising one or more adenosine deaminase (ADA) inhibitor and one or more xanthine oxidase (XO) inhibitor.

20. The composition of claim 19 wherein said ADA inhibitor is a compound selected from the group consisting of Formula I, Formula II, and Formula III.

21. The composition of claim 20 wherein said ADA inhibitor is pentostatin (dCF).

22. The composition of claim 19 wherein said XO inhibitor is a compound of Formula IV.

23. The composition of claim 19 wherein said compound of Formula IV is allopurinol.
